SEQUENCE LISTING (18 sequences)

SEQ ID NO: 1 - protein, 567 aa, Thermosporothrix hazakensis
MSGTTKRRPLRVVMMVLVLLSVLGLSMLPLNMNRVEAAGSGYWHTDGSRILDANNQPVRIAGINWFGFETANYTVHGLWTRNYRDMLDQIKSLGYNTIRLPYSNQLFDAGSTPTGIDFAKNPDLQGLTGLQIMDKIINYSGQIGLRIILDRHRPDSGGQSALWYTSAYPESRWLSDWKMLASRYKGNTTVIGADLHNEPHAPACWGCGDTSLDWRLAAERAGNAILSVNPDWLIFVEGVDCYVSGGGTNGGCYWWGGNLTGAQDYPVRLSVPGRLVYSAHDYPSSVYPQSWFSDPNYPNNLPALWDQRWGYLHKQGTAPVLLGEFGTKLQSTSDQQWLSKLTQYLGNGTSGMHWTFWSWNPNSGDTGGILNDDWTTVNQAKQAYLNPILFPLDGGNGGGTPTPTPSPTTTVTPPPTSVSLQIKYKDGAASNTSTNSLRPQLQIVNTGNTPINLADVTIRYWYTTEGGGTQAYTCDYATIGCSTVHGKFVTVSPGRTGADTYLEVSFTSGTLAPGKDTGELQQRVNKSDWSNYDQSNDYSRNGSFTTYTSWNKVTAYYKGALVWGTEP

SEQ ID NO: 2 - protein, 407 aa, Thermosporothrix hazakensis
MSVAHALLRVVLVLGCVLPVFAAGEAHAETMTPYEALARGPYTILGNQVLDASGKPYLFHGITRSGPELDCTGKQSPYDRSHLALMGVPVPNVADIKDGRYWGGNTVRVPLSQNFWLKGDMKISTCTAAGYRAFVHRLVDDLTALGLNVILNLHWSGAGGQVGGAGAEQQMPDTDAVPFWEQVAQTYKDYSNVLFELYDEPGISYQQGSCWKFGCMIVGDEVRVHYCGCFKLFSYQAVGMQTLLETIRKTGARNLVIANGTNGGYSLKQLSMYALEGTNVLYSSHPSNNASDKMPSFWTDHFGQFADRYPLLITAFGQYNCKADFVNMLFDYLDARQIGWIARAWFVSSTSRSTICAYPQLVTDYNGTPSHAMGESVYERLRDYHADALVRWKLAVSEMRSFKKSTR

SEQ ID NO: 3 - protein, 531 aa, Thermosporothrix hazakensis
MKGFFKPVARCMLFVMLTGMTFLTGLTVLSSQQKTAHAAENFVYRCGTHFCLGDRYFYFAGANTYDVFTYGDGSSSSTPDDIENKYMDKAKIDAHMAALQSDGVSVLRLWMFSHETWHGFEPSKGVYNEAEFALFDYIIQSAKAHNIRLLPTLENYWTAYGGIDTRLQWEGLPTGDANRWMFFNKTKCPGCFTQYKNYVHYVLNRVNHYSGVAYKDEPTIFAWELMNEPRYQNATPNENSTGTTLRAWVDEMASYIKSIDSHHMVGTGIEGHQAKYGFGGDEGNPFVYLQQSPFIDFTSAHPYPTEEWAHLSLDQTKQLIDAWVNDSHNVIGKPFFMGEFNVKGVDRSTWWREIYGELERLDVAGSAFWWYQATNVDSTYGVSKGAPELAVFRQHSANQQAKNVPINATPTPGITPTVSPTITPTPGATCSVRYTIESQWPDGFTGKIKITNNGSSTINGWTLAFSFAAGQKVQQGWSATWSQSGANVTVTNASWNGTIAPSGSVEIGFNGSWKGSNPVPATFTLNGTVCQ

SEQ ID NO: 4 - protein, 937 aa, Thermosporothrix hazakensis
MFAQTWKRALRYGLLLSMGLSMLVSALSIPTHPAKAAPAFNYAEALQKAILFYEAQQSGKLPSWNRLSWRGDSALDDGKDVGHDLTGGWFDAGDHVKFGLPMAFSATMLAWGVLEYGDAYQKSGQMTHILNNLHFVNDYFIKAHTAPNELWGQVGDGGPDHAWWGPAEVMPMKRPAYKIDASCPGSDLAGETAAAMAAASMVFRSSDATYADTLLTHAKQLYTFADTYRGKYDACIPAGGFYTSWSGYNDELVWGALWLYQATKDSTYLTKAEQYYANLSTEPQTTIKSYKWTIAWDDTSYGAYVLLAKLTGKQQYKDDAQRWLDFWTVGVNGQKITYSPGGEAFLSEWGSLRYAANTAFVALVYADYLGSSDPLYSRYHDFGVRQINYALGDNPRNCSYVVGFGACPPQDPHHRTSHGSWTDSLQNPTHNRHILYGALVGGPKAANDQYTDDRTDYTGNEVATDYNAAFTGALARLYKEFGGTPVTSMPDKPKDDDELYVMAGINAEGSTFTEIKALFINKTGWPARATSTLSLRYYFTLENGVTPDQISVTTNYTQCGNNVSRPTQVSGNLYFITVTCNAKLYPGGQDAYKKEVQFRINSAGSWDPKNDWSYQNLTKDVVKFDHIPLYESEKKVWGNEPVDTGAAPTVSITSPKDGSNFKPAPATVAIEATASDSDGQITKVEFYNGSTLLGSDTSAPYSYSWANVPEGSYTLTAKAYDNAGNSTTSSPIAISVGQAVPTVSITSPANNASFNAPASITITANASSAGGSITKVEFYNGSTLLGSDTSAPYSYSWANVPEGSYTLTAKAYDDAGGTATSAAVKITVKKAGVCSVKYDIANQWSNGFTASVTISNPGSTAINGWTLVFTFPNNQRITNIWNATMTQSNGQVTVKDAGYNATIAPNGSVTFGFNGEWSGSNGKPTSFILNGQSCTVE

SEQ ID NO: 5 - protein, 279 aa, Thermosporothrix hazakensis
MYAFRHKLGMFGMICLALLCALFVPTATALAETRTGPNDTIAFGKYFVQNNQWGKQYNNWGDGYQSITHDASQDGSGAWSTDFHWWNVLSDDAWHIKAWPSIVCGWQWGSWSNNSGLPVHLWDNKNVVTSWHFRMNGGSSYRADAAYDLWLHDESDWYWPTDEIMIWPWWTDEETGAHNGTHIATVTIGGATWDVYKDWASNAQSPRGGWTFWKFIRQGTTTQIDGLNIKDFLMYLQWGLPDGVERVPNARYLTSIQAGSEIWYGNGWFATDLFSVDIS

SEQ ID NO: 6 - protein, 468 aa, Thermosporothrix hazakensis
MTMRVGSGIRVLIVLALALGFLSMTSLPAKAAEBVCTVDGTIDNLGKYWLNNNLWGSNTGSGTQCTWDTSISGSTLAWGTRWNWTGEQNSVKSYASAVLGWHWGWKNPNTGLPVQVSQNTSVASNWSFTLTGNSDNRMNVSYDLWFHPTANPGNVNPSDELMVWLYKSGSIQPVGSRQATVTIAGTTWELWRGNAGWNVFSFVRTSSVTSASLDLRDFINDLVTRGWMDPSKYLISVEAGTEIFTGSGQLDTTAYSVEVGGSSGGNHPPAVSLTKPANGASFTAPASIDLAADASDSDGSISKVEFYSGSTLLSTDTSAPYTYTWGNVAAGSYTLTAKAYDNTGAVTTSAPVTVTVGGSGSGATCSVKYSVQNQWDTGFTAQVSITNNGSSAINGWRLGWTWAGNQRITNAWNATTSQNGNQVTATNASYNATIAAGGSVSFGFNGSYSGSNPQPSAFTLNGNPCSVN

SEQ ID NO: 7 - DNA, 1704 bp, Thermosporothrix hazakensis
atgtcagggacgacgaaaagacgaccattgcgcgtcgtcatgatggtacttgttttattgagcgttcttggattgagtatgttaccgttgaacatgaacagagttgaagcagcaggatcagggtactggcacactgacggcagccggattctcgatgccaacaatcagccggtgcgcattgcagggattaactggtttggctttgagacagcgaactataccgtgcatgggctctggacgcgcaactatcgcgacatgctggaccagatcaaaagcctgggctataacacgattcgcctcccctattcaaaccagctttttgatgcgggaagcacaccaaccggcatcgactttgccaaaaacccggatcttcaggggctgaccggcctgcaaattatggataagattattaactactccggtcagatcgggctacgtattattctggaccgacaccgcccggactcgggaggacagtcggcactctggtacacctcggcctatcctgaatcgcgctggctctccgactggaaaatgctcgcttcgcgctataaaggaaacacgacggtgatcggcgcggacctgcataacgagccacacgctcccgcctgttggggctgtggcgatacctcgcttgactggcggcttgcggctgaacgggccggaaacgcgattctctcggtcaacccggactggctgatctttgtcgagggcgttgattgctatgtgtccggtggcggcacgaatggcggctgctactggtggggcggcaacctgacaggcgcgcaagattatccggtacggctctctgtacccggacggctggtctattctgcgcacgactacccctcttcggtctacccgcagagctggtttagcgatcccaactacccgaacaacctgcctgccctgtgggaccagcgctggggctacctgcacaagcaaggcaccgcgcccgttctgcttggtgagtttggcaccaagctgcaaagcaccagcgatcagcaatggctcagcaaattgacgcagtatcttggcaatggcaccagcgggatgcactggaccttctggtcatggaaccccaattctggcgatacgggcggtattctcaacgacgactggactacagtcaatcaggcgaaacaggcctacctgaacccgattctgttcccgctggatggcggcaatggtggcgggacgcctactcccacgccaagcccgaccacgacagtgactccgccacccacttcggtctcactccagatcaagtataaggatggcgcagccagcaacaccagcacaaatagcctgaggcctcagcttcagatcgtgaataccggcaatacgccgatcaatctggcagatgtaaccattcgctactggtacactaccgaaggtggtggcactcaggcgtatacctgcgactatgcgaccattggttgcagcaccgttcacgggaaatttgtcacggtctcgccgggtcgtacaggcgcagatacctacctggaagtcagcttcacctcgggcacgctggcaccgggcaaggataccggggagcttcagcagcgcgttaacaagagcgactggtccaattacgaccagagcaatgactactcgcgcaacggctccttcactacctacactagctggaataaggtcacggcctactataaaggggcgctggtctggggtacagagccgtag

SEQ ID NO: 8 - DNA, 1224 bp, Thermosporothrix hazakensis
atgtctgttgctcatgctctactgcgtgttgtgctggtgcttggctgtgtgttgcccgtatttgctgccggcgaggctcacgcggagactatgaccccctatgaggccctggcaagaggcccttataccattctgggaaatcaggtgctggatgccagtgggaagccctatctctttcatggtatcacgcgttcagggccagaacttgattgtacaggcaagcagagcccttatgatcgttcgcatcttgccttgatgggtgtccctgtgcccaatgtggccgatatcaaagacgggagatattggggaggaaatacggttcgggtgccgctttctcagaatttctggctgaagggcgatatgaaaatttcgacctgtacggcggcggggtatcgagcatttgtgcaccgtctggttgacgatctgacagcactcggattgaatgtcattctgaacctgcactggtccggtgcgggtggacaggttggcggcgcaggtgcagaacagcaaatgcccgataccgatgcggtgccgttctgggaacaggtggcgcagacctacaaggactattccaacgtgcttttcgagctctatgatgagcccggtatctcgtatcagcaggggtcctgctggaaattcggctgtatgatcgtgggggatgaggtcagggtgcattattgcggctgcttcaagctcttttcctaccaggcagtggggatgcagaccttgctggaaacgattcgcaaaacgggagcccggaatctggtgattgcaaatggcaccaatggtgggtatagcctgaagcagctttccatgtacgcgctggaaggaaccaatgtcctttacagcagccatccctctaacaacgcgagcgataaaatgccttccttctggaccgaccatttcggccagttcgcggatcgttatccgttgttgattacagcatttggccagtacaattgcaaagccgactttgtgaacatgctttttgactatctggatgcgcgtcagattggctggattgctcgggcctggtttgtctcctctacgtctcggagcactatctgtgcctatccacaactggtaactgattacaatggcacgccatcacatgctatgggtgagagtgtctatgagcgtctacgcgattatcatgcggatgcgctcgttcgctggaaattggctgtaagtgagatgcgatctttcaagaaaagcacgcgctag

SEQ ID NO: 9 - DNA, 1596 bp, Thermosporothrix hazakensis
atgaaaggattcttcaaaccagtagcacgatgcatgctctttgtgatgcttacaggaatgaccttcctgactggcctgaccgtcttgtcgtcgcaacagaaaacagctcacgcagcggaaaattttgtgtaccgctgtggtacacacttctgcctgggtgatcgctacttctactttgcgggcgccaacacctacgacgtgttcacctacggagatggaagcagctcttcaacacctgatgatatcgagaataagtatatggacaaggcgaaaatagacgcccatatggccgcactgcaaagtgacggtgtttccgtgttgcgcctctggatgttcagtcatgagacgtggcatggtttcgagccgtcaaaaggcgtctataacgaggccgagtttgccctgtttgactacatcattcaatccgcgaaggcgcataatatccgcttgctgcccacgctggaaaactactggacggcctacggcggtattgatacgcgtctacaatgggaggggctacccacgggcgatgcgaaccgctggatgttcttcaataagaccaaatgccccggctgcttcacgcagtataagaactacgtgcactatgtgctgaatcgcgtcaatcactatagcggcgtggcgtataaagatgagccgactatttttgcctgggagctgatgaatgagccgcgctatcagaacgctacaccaaacgagaactcaactggaaccacgctgcgcgcctgggtagatgagatggcgtcgtacatcaagagcatcgattctcaccatatggtcggaaccgggattgaagggcatcaggcgaagtatggcttcggcggggatgaagggaatccctttgtctacctgcaacaatcgccctttattgacttcacgtccgctcacccctatcctaccgaggaatgggcacatctcagccttgatcagaccaaacagttgatcgatgcctgggtgaatgattcgcataacgtgattggaaagcccttcttcatgggcgagttcaacgtcaagggagtggatcgctcgacgtggtggcgcgagatttacggggaattggagcgtcttgacgtggccggctcggctttctggtggtatcaggcgacgaatgtagacagcacctacggggtctcgaaaggtgctcccgaactggccgtattccgtcagcactcggcgaaccagcaggccaagaatgtgcccattaacgcaacgccgacccctggcatcacgccaacagtttccccgaccatcacgccgactcctggcgcaacctgtagcgtgcgctatacgattgagagccagtggccggacggctttaccgggaaaatcaagatcacaaacaatggctcaagcaccatcaatggatggacccttgctttcagcttcgcggcaggccagaaggtgcagcagggatggtcggcgacctggtcacagagtggcgcaaacgtcaccgttaccaatgcttcatggaacgggacgattgcgccttctggttcggttgaaatcggcttcaatggctcctggaaggggagtaaccctgttcctgcaacctttacgttgaatggcaccgtttgccagtaa

SEQ ID NO: 10 - DNA, 2814 bp, Thermosporothrix hazakensis
atgttcgcgcaaacgtggaaacgcgctctcagatacgggctgctcctcagcatgggtctgagtatgctcgtcagcgcgctttccatcccgacccacccggcaaaggcagcaccggcattcaactatgccgaagcgttgcaaaaagcgattctcttctacgaagctcagcagtcggggaagcttccttcctggaaccggctctcatggcgcggtgactccgctctcgatgatggcaaggacgtaggtcacgatctgaccggaggatggttcgacgcaggtgatcacgtcaagttcggcctgcctatggccttttctgctaccatgctcgcctggggtgttcttgaatatggggatgcctatcaaaaaagcgggcagatgacccacatcctcaacaacctgcactttgtcaatgactattttatcaaagcacataccgcgcccaacgagctctgggggcaagtgggtgacggtggtccagatcatgcgtggtgggggcccgccgaggtcatgccgatgaagcgccccgcctacaagatcgatgccagttgccccggtagcgatctggcaggtgaaacagcagccgcgatggccgccgcctcgatggtcttccgttccagcgatgccacctatgccgatacactgctgacccatgccaaacagctctacaccttcgccgacacctatcgaggcaagtatgatgcatgcatccctgctggcggcttctacacctcgtggagtggctataatgacgaactcgtctggggtgcgctctggctctatcaggcaacaaaagactccacctatttaactaaagccgaacagtactacgcaaatctgagtactgagccacaaacaaccatcaaatcctacaaatggaccattgcatgggatgatacatcctatggcgcctacgtactgcttgctaaactgacaggtaaacagcagtataaggatgatgcacagcgctggctcgacttctggacggttggtgtcaatgggcagaaaatcacctattctccgggcggcgaggccttcttgagcgagtggggttctctacgttatgccgcgaataccgcttttgtcgctctggtctatgccgattatctgggaagcagcgatccgctctatagccgctaccacgacttcggcgtcagacagatcaactacgcgcttggtgacaatccgcgcaactgtagctacgtcgttggcttcggggcctgccctcctcaagatccacatcaccgcacctcacacggctcgtggactgactcgctacaaaatccaacgcataaccgccatatcctctatggtgcgctggtcggtggccccaaagccgccaacgatcagtacaccgatgatcgaaccgattacaccggaaatgaagtcgcgaccgactataatgcggccttcacaggtgcactggcacggctctacaaggagttcggaggcactccggtaacatccatgcccgataagccaaaagatgacgacgaactctacgttatggccggaatcaacgccgagggctcgactttcaccgagattaaggccctctttatcaacaagaccggctggcctgctcgcgccaccagtacgctttcactgcgctattacttcacgctggaaaatggcgtgacgcccgatcaaatcagcgtcaccaccaactacacccagtgtggaaataatgtctccaggccgacgcaggtttcgggcaatctctacttcatcactgttacatgtaacgcgaaactctatcctggtggacaggacgcttataaaaaagaggtacaattccgcatcaacagtgccggatcctgggaccccaaaaatgactggtcttatcaaaacctgaccaaagacgtcgttaaatttgaccatatcccgctgtacgaaagcgagaaaaaggtctggggtaacgagccagtcgacacaggcgcggcacccaccgtcagcatcaccagcccgaaagatggcagcaacttcaaacccgcgccggcaacggtcgcgattgaggctaccgccagcgatagcgacggacagatcacaaaggtggagttctacaatggctcgaccctcctgggcagcgacaccagcgctccatacagctatagttgggctaacgtaccagagggcagctacactctaaccgccaaggcgtatgacaatgctggaaatagcaccacttcttccccgatcgcgatcagcgtcgggcaggcggtgcccacagtcagcattacaagcccggctaataacgcaagctttaatgctcctgccagcatcacgattactgcaaacgcgagcagcgccggaggctctattaccaaagtagagttctataatggctcgacccttctgggcagcgacaccagcgctccatacagctatagttgggccaacgtaccagagggcagctacacactgaccgcgaaagcatatgatgatgcaggaggaacagcaacatcagcagcagtgaagataacagtcaaaaaagcgggtgtctgttcggtgaaatatgacatcgcgaatcagtggagcaacggcttcactgccagcgtcactatcagcaatccgggaagcacggctattaacggctggacgcttgtcttcaccttccccaacaatcagcgcatcaccaatatctggaatgcgacgatgacccagagcaatggccaggtcacggtgaaagatgcaggctacaacgccaccatcgcgccaaacggttccgtcaccttcggcttcaatggcgaatggagcggctccaatggcaagccgacaagctttatcctcaacgggcagagctgtaccgtagaataa

SEQ ID NO: 11 - DNA, 840 bp, Thermosporothrix hazakensis
atgtacgcttttcgtcacaaactcgggatgtttggtatgatctgtctggccttgctttgtgcgctgtttgtgccgactgcaaccgctttggccgaaaccagaaccggaccgaatgacacgattgcgttcgggaagtactttgtccagaacaaccagtgggggaaacagtacaacaactggggagatggctatcaaagcatcacccatgatgcgagtcaggacggttcgggagcctggtcaaccgatttccattggtggaatgtgctttctgatgatgcctggcatatcaaagcctggcccagcattgtgtgtggctggcaatgggggagctggtctaacaatagtggcctaccggtgcatctatgggataacaaaaatgtcgtcacaagctggcattttcggatgaatggtggtagctcatatcgggctgatgccgcgtatgatctctggttgcatgatgagagcgattggtattggcccactgatgagatcatgatctggccctggtggacagatgaggaaacaggcgcgcataacggcacccatatcgcgacggtgacgattggtggggcgacctgggatgtctacaaagactgggcctccaatgctcagagcccgcgcggtggctggactttctggaaatttatccgccaggggacaactacacagatcgacggcctgaacattaaggattttctgatgtatcttcagtggggcttgcccgatggcgttgaacgtgtccccaatgctcggtatctaaccagcattcaagccggaagcgaaatctggtatggcaatggctggtttgcgaccgatctcttctccgttgacatctcgtga

SEQ ID NO: 12 - DNA, 1404 bp, Thermosporothrix hazakensis
atgactatgcgagtaggctcgggcatacgcgtgcttatcgttctcgcgctggctctgggcttcctctcgatgacctctctaccggccaaagcggcggaagtgtgtacagttgatggcacgatcgacaatctcgggaagtactggctcaacaacaacctgtggggcagcaatacaggttcgggcacccagtgcacctgggatacatccatctctggctcaactctggcatggggaacgcgctggaactggaccggcgagcagaattctgtgaaatcgtatgccagcgcggtgcttggttggcactggggctggaagaatcccaataccggcttgcctgtgcaggtctcgcagaataccagcgtggcgagtaactggagctttaccctgaccggcaacagcgataaccggatgaatgtctcctatgatctctggttccatcctaccgcaaatcctggcaatgtgaacccttctgatgagctgatggtctggctatacaagtcaggctcgatccagccagttggctcgcgtcaggcaaccgtcactatcgccgggacgacctgggaactctggcgcggaaacgctggctggaacgtcttctcgttcgtcaggacatcaagcgtcacctcggcctcgttggatctgcgcgactttatcaatgatctggtaacacgtggctggatggacccgagcaagtatctgatcagtgtcgaggcgggaactgagatctttacaggaagtggtcagctcgatacaaccgcgtatagcgtcgaggttggcggcagcagtggcgggaatcatccgccagccgtgagcctgacaaagcccgccaatggggccagctttaccgccccggcctcgattgatctggccgccgatgcgagtgatagcgatggctctatcagcaaagtcgagttttacagcgggagcacgctgctgagcacagataccagcgccccctacacctacacctggggaaacgtggcggctggctcctatacactgaccgcgaaggcctacgacaacaccggagccgttacgacgtcagcgccggtaacggtgacagtgggtggctcaggtagtggcgcgacctgctcggtgaaatatagcgtgcagaaccagtgggatacagggtttacggcacaggtcagtatcaccaacaacggcagcagcgcaatcaatggctggcggctaggctggacctgggccggcaaccagcggattaccaacgcctggaacgctactaccagccagaatggcaatcaggtgacggcaaccaacgccagctataacgcgaccattgcggctggtggctcggtaagcttcggctttaatggctcctacagtggctcgaaccctcagccgagcgcgtttaccctgaatggcaatccctgtagtgtcaattga

SEQ ID NO: 13 - DNA, 23 bp, Artificial Sequence
  synthetic primer
atgtcagggacgacgaaaagacg

SEQ ID NO: 14 - DNA, 23 bp, Artificial Sequence
  synthetic primer
cggctctgtaccccagaccagcg

SEQ ID NO: 15 - DNA, 23 bp, Artificial Sequence
  synthetic primer
atgttcgcgcaaacgtggaaacg

SEQ ID NO: 16 - DNA, 23 bp, Artificial Sequence
  synthetic primer
ttctacggtacagctctgcccgt

SEQ ID NO: 17 - DNA, 27 bp, Artificial Sequence
  synthetic primer
atgactatgcgagtaggctcgggcata

SEQ ID NO: 18 - DNA, 28 bp, Artificial Sequence
  synthetic primer
attgacactacagggattgccattcagg

(19) United States
(12) Patent Application Publication
(10) Pub. No.: US 2013/0252284 A1
(43) Pub. Date: Sep. 26, 2013

(54) NOVEL CELLULASE DERIVED FROM THERMOSPOROTHRIX HAZAKENSIS

(71) Applicants: Shuhei Yabe, Miyagi (JP); Yoshifumi Aiba, Miyagi (JP); Yasuteru Sakai, Miyagi (JP); Akira Yokota, Osaka (JP)

(72) Inventors: Shuhei Yabe, Miyagi (JP); Yoshifumi Aiba, Miyagi (JP); Yasuteru Sakai, Miyagi (JP); Akira Yokota, Osaka (JP)

(73) Assignee: Kennan Eisei Kogyo Co., Ltd., Miyagi (JP)

(21) Appl. No.: 13/821,362

(22) PCT Filed: May 31, 2012
(86) PCT No.: PCT/JP2012/064164

§ 371 Date: Mar. 7, 2013

(30) Foreign Application Priority Data
Jun. 1, 2011 (JP) 2011-123754

(51) Int. Cl. C12N 9/24; C12P 7/14; C12P 19/14

(52) U.S. Cl. CPC C12N 9/2437; C12P 19/14; C12P 7/14
USPC 435/99; 435/209; 536/23.2; 435/320.1; 435/252.33; 435/254.21; 435/348; 435/365; 435/358; 435/369; 435/162

(57) ABSTRACT

This invention provides a novel cellulase derived from Thermosporothrix hazakensis. The cellulase derived from Thermosporothrix hazakensis has enzyme activity on at least β-glucan, soluble cellulose, crystalline cellulose, phosphoric acid-swollen cellulose, and xylan.

### TECHNICAL FIELD

The present invention relates to a novel cellulase derived from Thermosporothrix hazakensis. More particularly, the present invention relates to a novel cellulase derived from the Thermosporothrix hazakensis SK20-1^{T} strain (JCM 16142T=ATCC BAA-1881T).

### BACKGROUND ART

The term “cellulase” is a generic name referring to a group of enzymes that catalyze enzyme reaction systems for hydrolyzing cellulose into glucose, cellobiose, and cellooligosaccharide. Such enzymes are classified as exo-β-glucanase, endo-β-glucanase, β-glucosidase, or the like, depending on action mechanisms thereof. Through interactions between such enzymes which are cellulase, cellulose is degraded into glucose in the end.

In recent years, utilization of ethanol, lactic acid, or another substance as a liquid fuel or chemical raw material has drawn attention and been examined. Such substances are obtained via enzymolysis and saccharification of biomass resources with the use of cellulase, degradation thereof into a constitutional unit (i.e., glucose or xylose), and fermentation thereof.

However, the speed of cellulose degradation mediated by cellulases that have heretofore been utilized is not sufficiently high. In the presence of ethanol, salt, or other substances, in particular, cellulase activity is lowered. Accordingly, a cellulase capable of performing enzymolysis and saccharification of biomass resources in an efficient and cost-effective manner has been desired.

Thermosporothrix hazakensis is a bacteria belonging to the order Ktedonobacterales within the class Ktedonobacteria in the phylum Chloroflexi, and it is an aerobic and Gram-positive bacteria. The present inventors had isolated the Thermosporothrix hazakensis SK20-1^{T} strain (JCM 16142T=ATCC BAA-1881T) and demonstrated the capacity thereof for degrading cellulose, xylan, and chitin (Non-Patent Document 1). Up to the present, however, there has not been any report to the effect that a cellulase derived from Thermosporothrix hazakensis has been obtained.
Non-Patent Document 1: Shuhei Y. et. al., International Journal of Systematic and Evolutionary Microbiology, 2010, 60, 1794-1801

### SUMMARY OF THE INVENTION

### Object to be Attained by the Invention

It is an object of the present invention to provide a novel cellulase derived from Thermosporothrix hazakensis.

### Means for Attaining the Object

The present inventors have conducted concentrated studies in order to attain the above object. As a result, they discovered a novel cellulase from the Thermosporothrix hazakensis SK20-1^{T} strain (JCM 16142T=ATCC BAA-1881T). This has led to the completion of the present invention.

The present invention includes the following.

[1] A cellulase derived from Thermosporothrix hazakensis having enzyme activity on at least β-glucan, soluble cellulose, crystalline cellulose, phosphoric acid-swollen cellulose, and xylan.

[2] The cellulase according to [1], wherein the Thermosporothrix hazakensis is the Thermosporothrix hazakensis SK20-1^{T} strain (JCM 16142T=ATCC BAA-1881T).

[3] The cellulase according to [1] or [2], which retains enzyme activity at a temperature of at least 10° C. to 80° C.

[4] The cellulase according to [1] or [2], which retains enzyme activity at a pH of at least 2 to 11.

[5] The cellulase according to [1] or [2], which retains enzyme activity in the presence of an organic solvent at 0% to 25% (v/v) or higher concentration.

[6] The cellulase according to [5], wherein the organic solvent is selected from the group consisting of toluene, acetone, chloroform, butanol, hexane, and DMSO.

[7] The cellulase according to [1] or [2], which retains enzyme activity in the presence of ethanol at 0% to 50% (v/v) or higher concentration.

[8] The cellulase according to [α] or [2], which retains enzyme activity in the presence of salt at 0% to 25% (v/v) or higher concentration.

[9] The cellulase according to any of [1] to [8], which comprises one or more hydrolases selected from the group consisting of hydrolases comprising polypeptides represented by the amino acid sequences below:

(I) a polypeptide comprising the amino acid sequence as shown in SEQ ID NO: 1, a polypeptide comprising an amino acid sequence having deletion, substitution, insertion, or addition of one or several amino acids in the amino acid sequence as shown in SEQ ID NO: 1 and having cellulase activity, or a polypeptide comprising an amino acid sequence having at least 90% identity with the amino acid sequence as shown in SEQ ID NO: 1 and having cellulase activity;

(II) a polypeptide comprising the amino acid sequence as shown in SEQ ID NO: 2, a polypeptide comprising an amino acid sequence having deletion, substitution, insertion, or addition of one or several amino acids in the amino acid sequence as shown in SEQ ID NO: 2 and having cellulase activity, or a polypeptide comprising an amino acid sequence having at least 90% identity with the amino acid sequence as shown in SEQ ID NO: 2 and having cellulase activity;

(III) a polypeptide comprising the amino acid sequence as shown in SEQ ID NO: 3, a polypeptide comprising an amino acid sequence having deletion, substitution, insertion, or addition of one or several amino acids in the amino acid sequence as shown in SEQ ID NO: 3 and having cellulase activity, or a polypeptide comprising an amino acid sequence having at least 90% identity with the amino acid sequence as shown in SEQ ID NO: 3 and having cellulase activity;

(IV) a polypeptide comprising the amino acid sequence as shown in SEQ ID NO: 4, a polypeptide comprising an amino acid sequence having deletion, substitution, insertion, or addition of one or several amino acids in the amino acid sequence as shown in SEQ ID NO: 4 and having cellulase activity, or a polypeptide comprising an amino acid sequence having at least 90% identity with the amino acid sequence as shown in SEQ ID NO: 4 and having cellulase activity;

(V) a polypeptide comprising the amino acid sequence as shown in SEQ ID NO: 5, a polypeptide comprising an amino acid sequence having deletion, substitution, insertion, or addition of one or several amino acids in the amino acid sequence as shown in SEQ ID NO: 5 and having cellulase activity, or a polypeptide comprising an amino acid sequence having at least 90% identity with the amino acid sequence as shown in SEQ ID NO: 5 and having cellulase activity; and

(VI) a polypeptide comprising the amino acid sequence as shown in SEQ ID NO: 6, a polypeptide comprising an amino acid sequence having deletion, substitution, insertion, or addition of one or several amino acids in the amino acid sequence as shown in SEQ ID NO: 6 and having cellulase activity, or a polypeptide comprising an amino acid sequence having at least 90% identity with the amino acid sequence as shown in SEQ ID NO: 6 and having cellulase activity.

[10] A polynucleotide encoding the cellulase according to any of [1] to [9].

[11] An expression vector comprising the polynucleotide according to [10].

[12] A transformant obtained with the use of the expression vector according to [11].

[13] A culture product obtained by culturing the transformant according to [11].

[14] A detergent composition comprising the cellulase according to any of [1] to [9] or the culture product according to [13].

[15] A method for saccharification of a carbohydrate-containing raw material comprising treating a carbohydrate-containing raw material with the cellulase according to any of [1] to [9], the transformant according to [12], or the culture product according to [13].

[16] A method for producing a food or feed product comprising treating a carbohydrate-containing raw material with the cellulase according to any of [1] to [9], the transformant according to [12], or the culture product according to [13].

[17] A method for producing ethanol comprising:

(i) treating a carbohydrate-containing raw material with the cellulase according to any of [α] to [9], the transformant according to [12], or the culture product according to [13]; and

(ii) subjecting the product obtained in step (i) to fermentation.

This description contains part or all of the content as disclosed in the description and/or drawings of Japanese Patent Application No. 2011-123754, based on which the present application claims priority.

### Effects of the Invention

The present invention can provide a novel cellulase derived from Thermosporothrix hazakensis and, more particularly, a novel cellulase derived from the Thermosporothrix hazakensis SK20-1^{T} strain (JCM 16142T=ATCC BAA-1881T).

### BRIEF DESCRIPTION OF THE DRAWINGS

FIG. 1 shows the amino acid sequence of GH5-1 and the nucleotide sequence encoding the same.

FIG. 2 shows the amino acid sequence of GH5-2 and the nucleotide sequence encoding the same.

FIG. 3 shows the amino acid sequence of GH5-3 and the nucleotide sequence encoding the same.

FIG. 4-1 shows the amino acid sequence of GH9.

FIG. 4-2 shows the nucleotide sequence encoding the amino acid sequence of GH9.

FIG. 5 shows the amino acid sequence of GH12-1 and the nucleotide sequence encoding the same.

FIG. 6 shows the amino acid sequence of GH12-2 and the nucleotide sequence encoding the same.

FIG. 7 is a characteristic diagram showing enzyme activity of GH5-1, GH9, and GH12-2 on various substrates. In the table, “ND” stands for “not detected.” The substrate solution having a dark yellow color after enzyme treatment was designated as “++,” the substrate solution having a light yellow color was designated as “+,” and relative enzyme activity was evaluated. The amount of enzyme generating 1 mmol of a reducing sugar per minute (DRS) is designated as 1 unit (U).

FIG. 8 is a characteristic diagram showing the influence of enzyme activity of GH5-1, GH9, and GH12-2 depending on reaction temperature. The highest activity level of each enzyme is designated as 100%, and activity is indicated relative thereto (%).

FIG. 9 is a characteristic diagram showing the influence of enzyme activity of GH5-1, GH9, and GH12-2 depending on pH levels. The highest activity level of each enzyme is designated as 100%, and activity is indicated relative thereto (%).

FIG. 10 is a characteristic diagram showing the temperature stability of GH5-1, GH9, and GH12-2. The highest activity level of each enzyme is designated as 100%, and activity is indicated relative thereto (%).

FIG. 11 is a characteristic diagram showing organic solvent tolerance of GH5-1, GH9, and GH12-2. The highest activity level of each enzyme is designated as 100%, and activity is indicated relative thereto (%).

FIG. 12 is a characteristic diagram showing ethanol tolerance of GH5-1, GH19, and GH12-2. The highest activity level of each enzyme is designated as 100%, and activity is indicated relative thereto (%).

FIG. 13 is a characteristic diagram showing NaCl tolerance of GH5-1, GH9, and GH12-2. The highest activity level of each enzyme is designated as 100%, and activity is indicated relative thereto (%).

FIG. 14 is a characteristic diagram showing synergistic effects of enzyme activity attained with the use of GH5-1, GH9, and GH12-2 in combination.

FIG. 15-1 is a characteristic diagram showing the test results for substrate degradation performance of GH5-1, GH9, and GH12-2 via thin-layer chromatography (TLC). Each lane shows a sample treated with the substrate indicated below: C1: glucose; C2: cellobiose; C3: cellotriose; C4: cellotetraose; C5: cellopentaose; A.: phosphoric acid-swollen cellulose; Cr.: crystalline cellulose; G.: β-glucan; P.: filter paper; cmc: CM cellulose; and M: marker. The vertical axis represents the number of carbons in sugar.

FIG. 15-2 is a characteristic diagram showing the test results for substrate degradation performance of GH5-1, GH9, and GH12-2 via thin-layer chromatography (TLC). This indicates the number of carbons of degradation products observed when substrates are treated with enzymes.

### DETAILED DESCRIPTION OF THE PREFERRED EMBODIMENTS

The present invention relates to a novel cellulase derived from Thermosporothrix hazakensis. More specifically, the present invention relates to a novel cellulase derived from the Thermosporothrix hazakensis SK20-1^{T} strain (JCM 16142T=ATCC BAA-1881T) (hereafter, designated as the “SK20-1^{T} strain”).

The SK20-1^{T} strain was isolated from mature compost (Shuhei Y. et. al., described above) and registered as 16142^{T} with the Japan Collection of Microorganisms (JCM) and as BAA-1881T with the American Type Culture Collection (ATCC).

In the present invention, the cellulase uses at least β-glucan, soluble cellulose, crystalline cellulose, phosphoric acid-swollen cellulose, and xylan as substrates. Substrate specificity is described in detail in the “(1) Substrate specificity” section below.

In the present invention, the cellulase comprises one or more hydrolases selected from among the following. Such hydrolases are capable of cleaving non-crystalline regions in cellulose at random, and they have endo-hydrolase activity:

(I) a polypeptide comprising the amino acid sequence as shown in SEQ ID NO: 1, a polypeptide comprising an amino acid sequence having deletion, substitution, insertion, or addition of one or several amino acids in the amino acid sequence as shown in SEQ ID NO: 1 and having cellulase activity, or a polypeptide comprising an amino acid sequence having at least 90% identity with the amino acid sequence as shown in SEQ ID NO: 1 and having cellulase activity;

(II) a polypeptide comprising the amino acid sequence as shown in SEQ ID NO: 2, a polypeptide comprising an amino acid sequence having deletion, substitution, insertion, or addition of one or several amino acids in the amino acid sequence as shown in SEQ ID NO: 2 and having cellulase activity, or a polypeptide comprising an amino acid sequence having at least 90% identity with the amino acid sequence as shown in SEQ ID NO: 2 and having cellulase activity;

(III) a polypeptide comprising the amino acid sequence as shown in SEQ ID NO: 3, a polypeptide comprising an amino acid sequence having deletion, substitution, insertion, or addition of one or several amino acids in the amino acid sequence as shown in SEQ ID NO: 3 and having cellulase activity, or a polypeptide comprising an amino acid sequence having at least 90% identity with the amino acid sequence as shown in SEQ ID NO: 3 and having cellulase activity;

(IV) a polypeptide comprising the amino acid sequence as shown in SEQ ID NO: 4, a polypeptide comprising an amino acid sequence having deletion, substitution, insertion, or addition of one or several amino acids in the amino acid sequence as shown in SEQ ID NO: 4 and having cellulase activity, or a polypeptide comprising an amino acid sequence having at least 90% identity with the amino acid sequence as shown in SEQ ID NO: 4 and having cellulase activity;

(V) a polypeptide comprising the amino acid sequence as shown in SEQ ID NO: 5, a polypeptide comprising an amino acid sequence having deletion, substitution, insertion, or addition of one or several amino acids in the amino acid sequence as shown in SEQ ID NO: 5 and having cellulase activity, or a polypeptide comprising an amino acid sequence having at least 90% identity with the amino acid sequence as shown in SEQ ID NO: 5 and having cellulase activity; and

(VI) a polypeptide comprising the amino acid sequence as shown in SEQ ID NO: 6, a polypeptide comprising an amino acid sequence having deletion, substitution, insertion, or addition of one or several amino acids in the amino acid sequence as shown in SEQ ID NO: 6 and having cellulase activity, or a polypeptide comprising an amino acid sequence having at least 90% identity with the amino acid sequence as shown in SEQ ID NO: 6 and having cellulase activity.

The number of amino acids defined by the term “one or several” regarding the polypeptide is not particularly limited. For example, it is 20 or less, preferably 10 or less, more preferably 5 or less, and particularly preferably 4 or less. Alternatively, such number is 1 or 2.

The term “identity” regarding the polypeptide refers to the percentage of identical and similar amino acid residues relative to all the overlapping amino acid residues of two amino acid sequences that are aligned in an optimal manner with or without the introduction of gaps. Identity can be determined using a technique well-known in the art, such as sequence analysis software (e.g., BLAST (Basic Local Alignment Search Tool at the National Center for Biological Information, U.S.A.) using, for example, the default parameters; i.e., initial parameters). The term “at least 90% identity” refers to a homology of at least 90%, preferably 95%, and more preferably 99% or more.

The term “cellulase activity” regarding the polypeptide refers to activity of hydrolyzing cellulose into glucose, cellobiose, and cellooligosaccharide. In the present description, the “cellulase activity” may be referred to as “enzyme activity” or simply as “activity.” Cellulase activity can be measured in accordance with a conventional technique. For example, a known cellulose substrate (examples thereof include, but are not particularly limited to, filter paper, carboxymethyl cellulose (CMC), microcrystalline cellulose (Avicel), salicin, xylan, and cellobiose) is added to the polypeptide, the resultant is subjected to an enzyme reaction for a given period of time, the resulting reducing sugar is allowed to develop color with the use of the Somogy-Nelson method or the dinitrosalicylic acid (DNS) method, and colorimetry is then carried out at a given wavelength. Thus, cellulase activity can be measured. According to the Somogy-Nelson method, specifically, Somogyi's copper reagent (Wako Pure Chemical Industries) is added to a reaction solution that had been subjected to the reaction for a given period of time in order to terminate the reaction. The resultant is then boiled for about 20 minutes, and it is cooled with tap water immediately thereafter. After cooling, Nelson's reagent is added to dissolve the reduced copper precipitate, color is allowed to develop, the resultant is allowed to stand for about 30 minutes, distilled water is added thereto, and the absorbance is then measured. When the DNS method is employed, an enzyme solution is added to a 1% CMC substrate solution, the mixture is subjected to an enzyme reaction for a given period of time, and the enzyme reaction is then terminated via boiling or other means. Dinitrosalicylic acid is added to the reaction solution, the mixture is subjected to boiling for 5 minutes, the resultant is cooled, and the absorbance is then measured.

A particularly preferable cellulase in the present invention comprises one or more hydrolases selected from among the following.

(I) A polypeptide comprising the amino acid sequence as shown in SEQ ID NO: 1, a polypeptide comprising an amino acid sequence having deletion, substitution, insertion, or addition of one or several amino acids in the amino acid sequence as shown in SEQ ID NO: 1 and having cellulase activity, or a polypeptide comprising an amino acid sequence having at least 90% identity with the amino acid sequence as shown in SEQ ID NO: 1 and having cellulase activity;

(IV) a polypeptide comprising the amino acid sequence as shown in SEQ ID NO: 4, a polypeptide comprising an amino acid sequence having deletion, substitution, insertion, or addition of one or several amino acids in the amino acid sequence as shown in SEQ ID NO: 4 and having cellulase activity, or a polypeptide comprising an amino acid sequence having at least 90% identity with the amino acid sequence as shown in SEQ ID NO: 4 and having cellulase activity; and

(VI) a polypeptide comprising the amino acid sequence as shown in SEQ ID NO: 6, a polypeptide comprising an amino acid sequence having deletion, substitution, insertion, or addition of one or several amino acids in the amino acid sequence as shown in SEQ ID NO: 6 and having cellulase activity, or a polypeptide comprising an amino acid sequence having at least 90% identity with the amino acid sequence as shown in SEQ ID NO: 6 and having cellulase activity.

In the present invention, a more preferable cellulase comprises one or more hydrolases selected from among the following:

(Ia) a polypeptide comprising the amino acid sequence as shown in SEQ ID NO: 1;

(IVa) a polypeptide comprising the amino acid sequence as shown in SEQ ID NO: 4; and

(VIa) a polypeptide comprising the amino acid sequence as shown in SEQ ID NO: 6.

In this description, one or more hydrolases described in (I) to (VI) above may occasionally be referred to as “cellulase(s).”

The cellulase of the present invention has the characteristics described below.

### (1) Substrate Specificity

The cellulase of the present invention uses β-glucan, soluble cellulose (CM cellulose), phosphoric acid-swollen cellulose, crystalline cellulose, xylan, mannan, laminarin, para-nitrophenyl cellobioside, para-nitrophenyl glucoside, curdlan, dextran, mutan, arabinoxylan, chitin, galactan, galactomannan, pullulan, xyloglucan, or filter paper as a substrate, although substrates are not limited thereto. Preferably, the cellulase has enzyme activity on at least β-glucan, soluble cellulose, crystalline cellulose, phosphoric acid-swollen cellulose, and xylan.

In particular, the hydrolase (I) above has activity on substrates such as β-glucan, soluble cellulose (CM cellulose), phosphoric acid-swollen cellulose, crystalline cellulose, xylan, para-nitrophenyl cellobioside, and para-nitrophenyl glucoside.

The hydrolase (IV) above has activity on substrates such as β-glucan, soluble cellulose (CM cellulose), phosphoric acid-swollen cellulose, crystalline cellulose, xylan, and para-nitrophenyl cellobioside.

Further, the hydrolase (VI) above has activity on substrates such as β-glucan, soluble cellulose (CM cellulose), phosphoric acid-swollen cellulose, crystalline cellulose, and xylan.

In general, endo-cellulase derived from bacteria does not have activity on crystalline cellulose or phosphoric acid-swollen cellulose. Thus, the substrate specificity of the cellulase of the present invention can be regarded as a distinctive feature.

As described in detail in the examples below, the activity of such cellulase on CM cellulose is higher than that of a general cellulase that has been recently utilized at an industrial level (e.g., endo-cellulase derived from Trichoderma viride) by 1.5 to 6 times, and preferably by 2 to 4 times (Kayoko Hirayama et al., Biosci. Biotechnol. Biochem., 74 (8), 1690-1686, 2010).

### (2) Reaction Temperature Range

The optimal temperature at which the cellulase of the present invention exhibits its activity is 5° C. to 90° C., and preferably 10° C. to 80° C.

As described in detail in the examples below, in particular, the optimal temperature at which the hydrolases (I) and (VI) above exhibit activity is 5° C. to 90° C., and preferably 10° C. to 80° C. The optimal temperature at which the hydrolase (IV) above exhibits activity is 45° C. to 65° C., and preferably about 60° C.

### (3) pH Range

The optimal pH level at which the cellulase of the present invention exhibits activity is 2 to 11, and preferably 3 to 10.

As described in detail in the examples below, in particular, the optimal pH level at which the hydrolase (I) exhibits activity is 3 or more, and preferably 4 to 11. The optimal pH level at which the hydrolase (IV) exhibits activity is 3.5 to 9, and preferably about 4. Further, the optimal pH level at which the hydrolase (VI) exhibits its activity is 2 to 10.5, and preferably 3 to 9.

(4) Heat Tolerance The cellulase of the present invention has stability in heat treatment at 50° C. to 80° C., and preferably at 50° C. to 70° C. The term “stability” refers to the property of activity not being completely lost upon heat treatment. It does not necessarily mean that 100% of the activity before treatment is completely retained.

As described in detail in the examples below, in particular, the hydrolase (I) does not substantially lose its activity and remains stable upon thermal treatment at 70° C. within 30 minutes. The hydrolase (VI) maintains a high level of activity and remains stable as a result of thermal treatment at 70° C. for less than 10 minutes.

### (5) Organic Solvent Tolerance

The cellulase of the present invention can maintain its activity in the presence of an organic solvent at 0% to 80% (v/v), preferably 0% to 50% (v/v), and more preferably 0% to 25% (v/v) concentration. The term “organic solvent” used herein refers to one or more organic solvents selected from among toluene, acetone, chloroform, butanol, hexane, dimethyl sulfoxide (DMSO), ethylene glycol, 1,4-butanediol, 1,5-pentanediol, 1-hexanol, methanol, 2-propanol, triethylene glycol, dimethylformamide, and 1,4-dioxane, although organic solvents are not limited thereto.

As described in detail in the examples below, in particular, the hydrolase (I) above maintains a high level of activity in the presence of toluene, chloroform, hexane, or DMSO. Also, the hydrolase (IV) above maintains a high level of activity in the presence of hexane. The hydrolase (VI) above maintains a high level of activity in the presence of toluene, acetone, chloroform, or hexane.

The cellulase of the present invention can maintain a high level of activity in the presence of an organic solvent. Accordingly, it is very useful when cellulase treatment needs to be carried out in the presence of an organic solvent (e.g., a fine chemical application such as synthesis of sucrose fatty acid ester).

### (6) Ethanol Tolerance

The cellulase of the present invention can maintain activity in the presence of ethanol at 0% to 70% (v/v), 0% to 60% (v/v), preferably, 0% to 50% (v/v), and more preferably 0% to 30% (v/v) concentration.

As described in detail in the examples below, in particular, the hydrolase (I) above can maintain a high level of activity in the presence of ethanol at 50% (v/v) concentration, the hydrolase (VI) above can maintain a high level of activity in the presence of ethanol at about 30% (v/v) or lower concentration, and the hydrolase (IV) above can maintain a high level of activity in the presence of ethanol at about 15% (v/v) or lower concentration.

The cellulase of the present invention can maintain a high level of activity in the presence of ethanol, it can be subjected to saccharification simultaneously with alcohol fermentation of biomass materials, and it is thus very useful.

### (7) NaCl Tolerance

The cellulase of the present invention can maintain activity in the presence of salt at 0% to 25% (v/v) concentration.

As described in detail in the examples below, in particular, the hydrolases (IV) and (VI) above maintain a high level of activity in the presence of salt at 25% (v/v) or lower concentration.

The cellulase of the present invention can maintain a high level of activity in the presence of salt, and it is very useful when cellulase treatment needs to be carried out at high salt concentrations (e.g., saccharification, following neutralization of a ligneous biomass material treated with an acid or alkali).

### (8) Synergistic Effects of Combination

The cellulase activity of the hydrolases (I) to (V) above can be synergistically improved with the use thereof in combinations of two or more. The term “two or more” used herein refers to 2 or more, 3 or more, 4 or more, 5 or more, and 6 hydrolases selected from among the hydrolases (I) to (VI) above. With the use of hydrolases in combination, cellulase activity can be improved by about 2 to 50 times, compared with the activity attained with the use of a hydrolase alone.

### (9) Substrate Degradation Mechanism

The cellulase of the present invention is capable of degrading a substrate into glucose or oligosaccharide.

As described in detail in the examples below, in particular, cellotriose (C3) is the minimum unit that the hydrolases (I) and (VI) can degrade (the minimal degradation unit). Also, the minimal degradation unit of the hydrolase (IV) is cellotetraose (C4).

In addition, the cellulase of the present invention has transgrlycolation activity. As described in detail in the examples below, in particular, when the hydrolases (I) and (VI) are allowed to react with a trisaccharide or tetrasaccharide, they are capable of producing oligosaccharides with chains as long as or longer than such saccharides because of the transgrlycolation activity thereof.

In the present invention, the polypeptide may be a substance purified or roughly purified from the culture product or culture supernatant of the SK20-1^{T} strain. Alternatively, the polypeptide may be a substance purified or roughly purified from the culture product or culture supernatant of a gene recombinant transformant expressing the polypeptide, as described in detail below. The polypeptide can be adequately purified or roughly purified from the culture product or culture supernatant via a general protein purification technique, such as ammonium sulfate or ethanol precipitation, acid extraction, anion- or cation-exchange chromatography, reversed-phase high-performance liquid chromatography, affinity chromatography, gel filtration chromatography, or electrophoresis. Alternatively, the polypeptide may be chemically synthesized (peptide synthesis).

In the present invention, the polypeptide may be fixed to a solid phase. Examples of solid phases include, but are not particularly limited to, polyacrylamide gel, polystyrene resin, porous glass, and metal oxide. Fixation of the polypeptide to a solid phase is advantageous since it enables continuous and repetitive use of the polypeptide.

According to similarity and hydrophobic cluster analysis of amino acid sequences, the hydrolases (I) to (III) belong to the same enzyme family. The hydrolases (V) and (VI) belong to an enzyme family different from the enzyme family mentioned above, according to similarity and hydrophobic cluster analysis of amino acid sequences. Hydrolases of the same enzyme family can have similar properties in terms of, for example, substrate specificity, reaction temperature range, pH range, heat tolerance, organic solvent tolerance, ethanol tolerance, NaCl tolerance, synergistic effects of combination, and substrate degradation mechanisms. Accordingly, the properties of the hydrolases (II) and (III) can be similar or identical to those of the hydrolase (I). Also, the properties of the hydrolase (V) can be similar or identical to those of the hydrolase (VI).

The present invention also relates to a polynucleotide encoding the polypeptide. A polynucleotide encoding the polypeptide is selected from among the nucleotide sequences (i) to (vi) below.

### Nucleotide Sequence Encoding Polypeptide (I)

(i) The nucleotide sequence as shown in SEQ ID NO: 7; a nucleotide sequence consisting of a nucleotide sequence having deletion, substitution, or addition of one or several nucleotides in the nucleotide sequence as shown in SEQ ID NO: 7 and encoding a polypeptide having cellulase activity; a nucleotide sequence consisting of a nucleotide sequence hybridizing under stringent conditions to a nucleic acid comprising a sequence complementary to the nucleotide sequence as shown in SEQ ID NO: 7 and encoding a polypeptide having cellulase activity; or a nucleotide sequence having at least 90% identity with the nucleotide sequence as shown in SEQ ID NO: 7.

### Nucleotide Sequence Encoding Polypeptide (II)

(ii) The nucleotide sequence as shown in SEQ ID NO: 8; a nucleotide sequence consisting of a nucleotide sequence having deletion, substitution, or addition of one or several nucleotides in the nucleotide sequence as shown in SEQ ID NO: 8 and encoding a polypeptide having cellulase activity; a nucleotide sequence consisting of a nucleotide sequence hybridizing under stringent conditions to a nucleic acid comprising a sequence complementary to the nucleotide sequence as shown in SEQ ID NO: 8 and encoding a polypeptide having cellulase activity; or a nucleotide sequence having at least 90% identity with the nucleotide sequence as shown in SEQ ID NO: 8.

### Nucleotide Sequence Encoding Polypeptide (III)

(iii) The nucleotide sequence as shown in SEQ ID NO: 9; a nucleotide sequence consisting of a nucleotide sequence having deletion, substitution, or addition of one or several nucleotides in the nucleotide sequence as shown in SEQ ID NO: 9 and encoding a polypeptide having cellulase activity; a nucleotide sequence consisting of a nucleotide sequence hybridizing under stringent conditions to a nucleic acid comprising a sequence complementary to the nucleotide sequence as shown in SEQ ID NO: 9 and encoding a polypeptide having cellulase activity; or a nucleotide sequence having at least 90% identity with the nucleotide sequence as shown in SEQ ID NO: 9.

### Nucleotide Sequence Encoding Polypeptide (IV)

(iv) The nucleotide sequence as shown in SEQ ID NO: 10; a nucleotide sequence consisting of a nucleotide sequence having deletion, substitution, or addition of one or several nucleotides in the nucleotide sequence as shown in SEQ ID NO: 10 and encoding a polypeptide having cellulase activity; a nucleotide sequence consisting of a nucleotide sequence hybridizing under stringent conditions to a nucleic acid comprising a sequence complementary to the nucleotide sequence as shown in SEQ ID NO: 10 and encoding a polypeptide having cellulase activity; or a nucleotide sequence having at least 90% identity with the nucleotide sequence as shown in SEQ ID NO: 10.

### Nucleotide Sequence Encoding Polypeptide (V)

(v) The nucleotide sequence as shown in SEQ ID NO: 11; a nucleotide sequence consisting of a nucleotide sequence having deletion, substitution, or addition of one or several nucleotides in the nucleotide sequence as shown in SEQ ID NO: 11 and encoding a polypeptide having cellulase activity; a nucleotide sequence consisting of a nucleotide sequence hybridizing under stringent conditions to a nucleic acid comprising a sequence complementary to the nucleotide sequence as shown in SEQ ID NO: 11 and encoding a polypeptide having cellulase activity; or a nucleotide sequence having at least 90% identity with the nucleotide sequence as shown in SEQ ID NO: 11.

### Nucleotide Sequence Encoding Polypeptide (VI)

(vi) The nucleotide sequence as shown in SEQ ID NO: 12; a nucleotide sequence consisting of a nucleotide sequence having deletion, substitution, or addition of one or several nucleotides in the nucleotide sequence as shown in SEQ ID NO: 12 and having cellulase activity; a nucleotide sequence consisting of a nucleotide sequence hybridizing under stringent conditions to a nucleic acid comprising a sequence complementary to the nucleotide sequence as shown in SEQ ID NO: 12 and encoding a polypeptide having cellulase activity; or a nucleotide sequence having at least 90% identity with the nucleotide sequence as shown in SEQ ID NO: 12.

The number of nucleotides defined by the term “one or several” regarding the nucleotide sequence is not particularly limited. For example, it is 50 or less, preferably 20 or less, and more preferably 10 or less.

Under the “stringent conditions,” so-called specific hybrids are formed, but non-specific hybrids are not formed. For example, hybridization is carried out in a solution containing 2 to 6×SSC (1×SSC composition: 0.15 M NaCl, 0.015 M sodium citrate, pH 7.0) and 0.1% to 0.5% SDS at 42° C. to 55° C., and washing is then carried out in a solution containing 0.1 to 0.2×SSC and 0.1% to 0.5% SDS at 55° C. to 65° C.

The term “at least 90% identity” used with respect to the nucleotide sequence herein refers to identity of at least 90%, preferably at least 95%, and more preferably at least 99% determined using a technique well-known in the art, such as sequence analysis software (e.g., BLAST (Basic Local Alignment Search Tool at the National Center for Biological Information, U.S.A.) with reference to, for example, the default parameters; i.e., initial parameters).

The “cellulase activity” is as defined above.

The nucleotide sequence mentioned above includes that of a naturally-occurring mutant. Specific examples thereof include mutants based on polymorphisms such as SNPs (i.e., single nucleotide polymorphisms), splice mutants, and mutants resulting from degeneracy of genetic codes.

Alternatively, the nucleotide sequence may be modified in accordance with the codon frequency of a host organism to be transformed, which is described below in detail.

The present invention also relates to an expression vector comprising the polynucleotide.

By introducing the expression vector of the present invention into an adequate host cell, a hydrolase encoded by the polynucleotide can be expressed.

The expression vector of the present invention can be prepared via a genetic engineering technique well-known in the art. Specifically, the polynucleotide may be incorporated into a general vector for gene introduction and expression known in the art, and the expression vector of the present invention can be thus prepared. A plasmid, phage, virus, or other vector can be used as the expression vector of the present invention without particular limitation, provided that it is capable of replication in a host cell. Specific examples include: Escherichia coli plasmids, such as pBR322, pBR325, pUC118, pUC119, pKC30, and pCFM536; Bacillus subtilis plasmids, such as pUB 110; yeast plasmids, such as pG-1, YEp13, and YCp50; phage DNAs, such as λgt110 and λZAPII; and DNA or RNA viruses, such as retrovirus, herpesvirus, vaccinia virus, poxvirus, poliovirus, Sindbis virus, Sendai virus, SV40, and human immunodeficiency virus (HIV). A vector can comprise one or more types of polynucleotides selected from among the above (e.g., 2, 3, 4, or more types thereof).

A vector can comprise, in addition to the above polynucleotide, a replication origin that enables replication in a host cell, a selection marker that identifies a transformant, and, preferably, an adequate transcription or translation control sequence derived from the host cell ligated to the polynucleotide, according to need. Examples of control sequences include a transcriptional promoter, operator, or enhancer, an mRNA ribosome-binding site, and an adequate sequence that controls initiation and termination of transcription and translation. Any promoters capable of driving gene expression in host cells can be used without particular limitation. Promoters known in the art, such as Pol III promoters (e.g., T3 promoters, T7 promoters, U6 promoters, or H1 promoters), can be adequately used. Common selection markers can be used in accordance with conventional techniques. Examples thereof include genes tolerant to ampicillin, bleomycin, hygromycin, neomycin, and puromycin and uridine and arginine biosynthetic genes.

The present invention also relates to a transformant comprising the expression vector.

The transformant of the present invention can be prepared by introducing the expression vector into a host cell for transformation. The transformant of the present invention is not particularly limited, provided that it comprises the polynucleotide. For example, a transformant may comprise the polynucleotide incorporated into the chromosome of the host cell. Alternatively, a transformant may comprise a vector comprising the polynucleotide. Further, the polypeptide may or may not be expressed in a transformant.

The expression vector can be introduced into a host cell by the calcium phosphate method, the calcium chloride/rubidium chloride method, electroporation, electroinjection, chemical processing such as PEG, a method involving the use of a gene gun, or other techniques.

Cells well-known in the art, such as E. coli, yeast (Saccharomyces cerevisiae), SF9, SF21, COS1, COST, CHO, and HEK293 cells, can be used as host cells.

The transformant comprising the expression vector that has been introduced therein is capable of expressing the hydrolase. A culture product of the transformant may be used as the hydrolase in that state. Alternatively, an expressed hydrolase may be purified or roughly purified from a culture product of the transformant via a conventional protein purification technique, such as centrifugation, salting out with ammonium sulfate, separation via precipitation with an organic solvent (e.g., ethanol, methanol, or acetone), chromatography techniques, such as ion-exchange chromatography, isoelectric chromatography, gel filtration chromatography, hydrophobic chromatography, adsorption column chromatography, substrate- or antibody-based affinity chromatography, or reversed-phase column chromatography, or filtration, such as precision filtration, ultrafiltration, or reverse osmosis filtration. These techniques can be performed alone or in combinations of two or more.

Examples of “culture products” include, but are not limited to, culture supernatant, broken cells, transformants, lyophilized products of any thereof, and those fixed on solid-phases (as defined above).

The present invention also relates to a detergent composition comprising the polypeptide or a culture product of the transformant as a detergent component. Such detergent composition may be a solid or liquid, with a liquid being preferable.

The detergent composition of the present invention can comprise the polypeptide or a culture product of the transformant in an amount of about 0.001% to 10% by weight. The detergent composition can comprise, in addition to the polypeptide or a culture product of the transformant, a surfactant. The detergent composition can comprise a surfactant in an amount of about 1% to 55% by weight. A surfactant can be an anionic, nonionic, cationic, amphoteric, or zwitterionic surfactant, and a mixture of any thereof can also be used. Examples of surfactants that can be used in the present invention include, but are not limited to, linear alkylbenzene sulfonate, alkyl sulfate, alpha-olefin sulfonate, polyoxyethylene alkyl ether sulfate, alpha-sulfo fatty acid ester salt, alkali metal salt of natural fatty acid, polyoxyethylene alkyl ether, alkyl polyethylene glycol ether, nonylphenol polyethylene glycol ether, fatty acid methyl ester ethoxylate, fatty acid ester of sucrose or glucose, alkyl glucoside, and ester of polyethoxylated alkyl glucoside. The detergent composition of the present invention can further comprise other detergent components known in the art, such as a builder, a bleaching agent, a bleaching activatot, a corrosion inhibitor, a sequestering agent, a polymer capable of releasing soil, an aroma chemical, other enzymes (e.g., protease, lipase, and amylase), an enzyme stabilizer, a pharmaceutic aid, a fluorescent brightening agent, and a foaming accelerator.

The present invention also relates to a method for saccharification of a carbohydrate-containing raw material using the polypeptide, a culture product of the transformant, or a culture product of the transformant.

The term “carbohydrate-containing raw material” refers to any carbohydrate, such as a monosaccharide, oligosaccharide, or polysaccharide, or a material derived from an organism containing the same. Examples of carbohydrate-containing raw materials include, but are not particularly limited to, cellulosic and/or lignocellulosic biomass materials produced by plants or algae. Specific examples include, but are not limited to, used paper, remaining lumber, wood, wheat bran, wheat straw, rice straw, rice husk, bagasse, soymeal, soybean curd waste, coffee processing waste, rice bran, wheat straw, corn stover, and corn cob.

A carbohydrate-containing raw material can be subjected to saccharification in accordance with a conventional technique. For example, a carbohydrate-containing raw material roughly ground, chipped, or treated with an acid or alkali is suspended in an aqueous medium, the polypeptide, the transformant, or a culture product of the transformant is added thereto, and the mixture is heated with agitation or shaking. Thus, a carbohydrate-containing raw material can be saccharified. According to this technique, the pH level and temperature of the reaction solution can be adequately determined in such a manner that the polypeptide would not be inactivated. Such reaction may be performed in a batch or continuous system. Examples of a saccharification product of the carbohydrate-containing raw material obtained by the method described above include saccharides, such as glucose, fructose, and sucrose.

The saccharification product of the carbohydrate-containing raw material obtained by the method described above can be used as a raw material for a food or feed product.

The present invention further relates to a method for producing ethanol comprising subjecting the saccharification product of the carbohydrate-containing raw material obtained by the method described above to fermentation. The saccharification product can be subjected to fermentation in accordance with a conventional technique. For example, known microorganisms capable of alcohol fermentation may be cultured in a medium containing the saccharification product of the carbohydrate-containing raw material obtained by the method above (e.g., yeast, such as Saccharomyces cerevisiae, and bacteria, such as Lactobacillus brevis, Clostridium, Thermoanaerobium brockii, and Zymomonas). The pH level and temperature of a medium and the culture duration can be adequately determined in accordance with microorganisms to be used. After the completion of culture, the medium is collected, and ethanol is separated therefrom. Ethanol can be separated from the medium by a known technique, such as distillation or pervaporation, with separation via distillation being preferable. Subsequently, separated ethanol is further purified (ethanol can be purified by a conventional technique such as distillation), and ethanol can then be obtained. As described above, the polypeptide of the present invention can maintain a high level of activity in the presence of ethanol. Accordingly, the step for saccharification of a carbohydrate-containing raw material and the step for fermentation of a saccharification product can be simultaneously carried out in the method for producing ethanol of the present invention.

### EXAMPLES

Hereafter, the present invention is described in greater detail with reference to the examples, although the technical scope of the present invention is not limited thereto.

### Example 1

### Cloning of Novel Cellulase

### <Preparation of Chromosome DNA and Genome Decoding>

The Thermosporothrix hazakensis SK20-1 strain (TCM 16142^{T}=ATCC BAA-1811^{T}) was subjected to shake culture in a tryptone yeast extract broth (ISP1) medium (DIFCO) at 50° C. for 3 days. The cultured cells were harvested, washed three times in TE buffer, and suspended in 5 ml of Tris-HCl buffer. Achromopeptidase (2.5 mg, Sigma) and 2.5 mg of chicken albumen lysozyme (Sigma) were added thereto, and the mixture was allowed to stand at 37° C. for 3 hours. Thereafter, Proteinase K (10 U, Sigma) and 250 μl of 10% SDS solution were added, and the mixture was allowed to stand at 37° C. for 1 day. The equivalent amount of a phenol/chloroform/isoamyl alcohol solution (25:24:1, Nippon Gene Co., Ltd.) was added thereto, the mixture was agitated and centrifuged, and an aqueous phase was collected. This procedure was repeated until the intermediate layer disappeared. The obtained aqueous phase was subjected to RNase treatment and it was then precipitated with ethanol. Thus, 40 μg of chromosome DNA was obtained.

The genome sequence was decoded by the pair-end sequencing method using a GS FLX Titanium System (Roche) (¼ plate, 4-kb library). Genome decoding was requested for Macrogen Inc. The results demonstrate that the number of total reads was 227,774,565 bp, that of contigs of 100 bp or longer was 131, that of scaffolds was 11, and the redundancy was 32. That is, 99% or more of the genome sequence was decoded.

### <Detection of Novel Cellulase and Cloning>

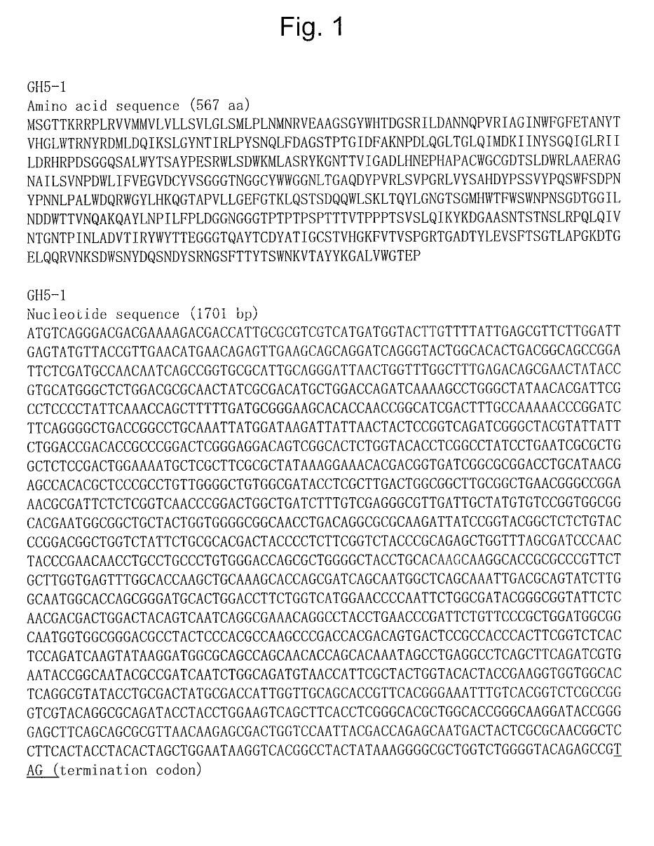

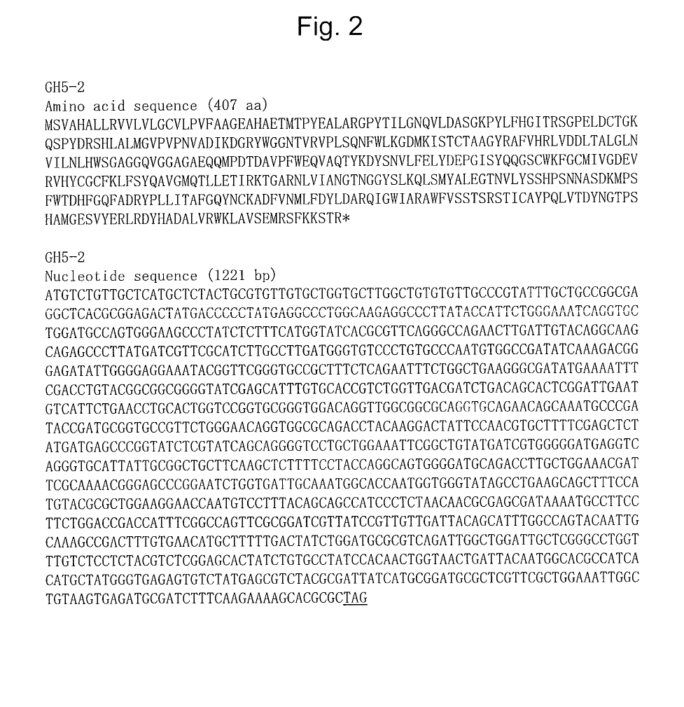

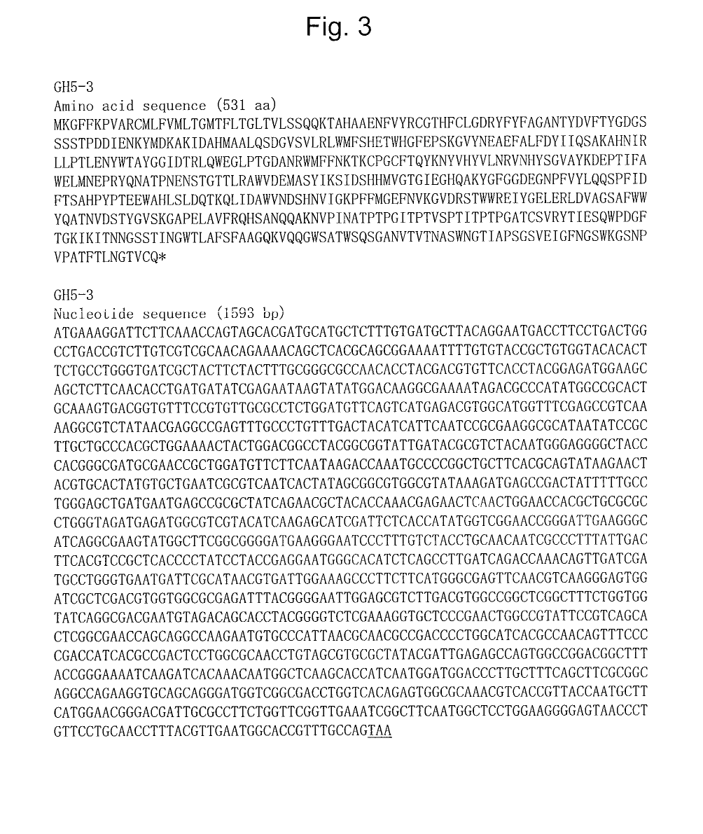

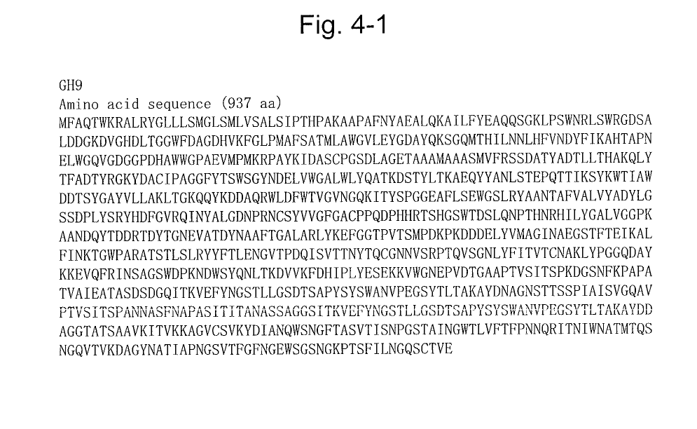

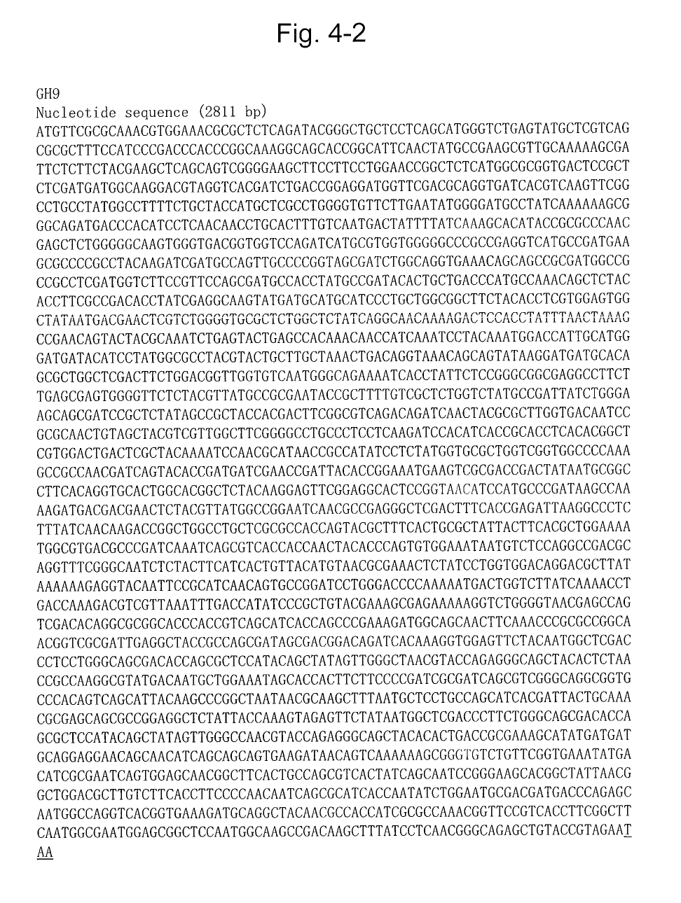

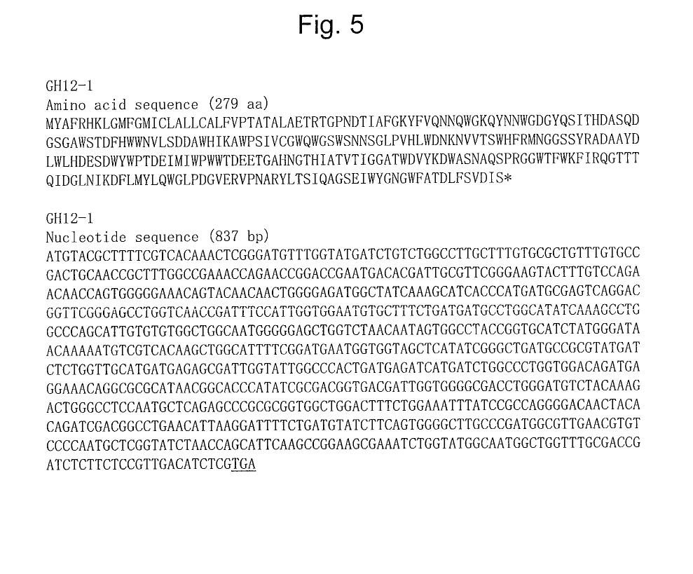

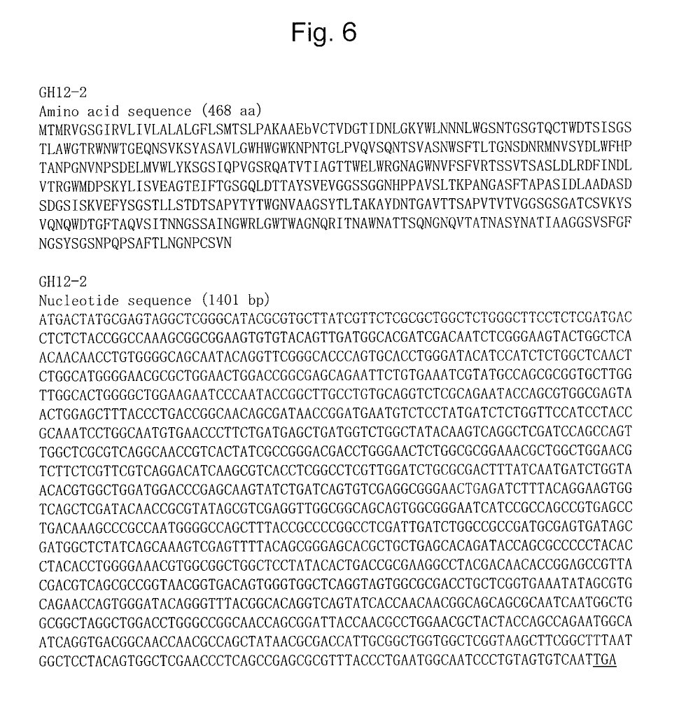

The obtained genome sequence was subjected to auto annotation using MiGAP (Microbial Genome Annotation Pipeline) (http://www.migap.org/). ORF search was carried out using Glimmer with reference to the TrEMBL (2010.7.13) and NCBI RefSeq (2010.7.21) databases. Sequences with identity of 30% or higher and coverage of 50% or higher were annotated. Glycoside hydrolase (GH) family search was carried out using the Carbohydrate-Active Enzymes database (http://www.cazy.org/Glycoside-Hydrolases.html). As a result, information regarding the translational regions of 6 cellulase genes (GH5-1, 5-2, 5-3, 9, 12-1, and 12-2) was obtained. FIG. 1 to FIG. 6 show the nucleotide sequences of the cellulase genes and the amino acid sequences encoded by such nucleotide sequences. The primers shown below were designed based on the information regarding the translational regions of the GH5-1, 9, and 12-2 genes.

Primers (GH5-1)
(SEQ ID NO: 13)
Forward: 5′-ATGTCAGGGACGACGAAAAGACG-3′
(SEQ ID NO: 14)
Reverse: 5′-CGGCTCTGTACCCCAGACCAGCG-3′
Primers (GH9)
(SEQ ID NO: 15)
Forward: 5′-ATGTTCGCGCAAACGTGGAAACG-3′
(SEQ ID NO: 16)
Reverse: 5′-TTCTACGGTACAGCTCTGCCCGT-3′
Primers (GH12-2)
(SEQ ID NO: 17)
Forward: 5′-ATGACTATGCGAGTAGGCTCGGGCATA-3′
(SEQ ID NO: 18)
Reverse: 5′-ATTGACACTACAGGGATTGCCATTCAGG-3′

With the use of the primer pairs shown above, PCR was carried out using chromosome DNA as the template in accordance with the compositions and programs shown below.

PCR reaction cocktail

### Chromosome DNA: 0.5 μl

0.2 mM forward primer: 1 μl
0.2 mM reverse primer: 1 μl
10×Taq buffer (TaKaRa): 5 μl
2.5 mM dNTPs (TaKaRa): 4 μl

### Taq (TaKaRa): 1 μl

Ion exchange water: 35.7 ml
PCR conditions

After heating was carried out at 95° C. for 2 minutes, a cycle of 95° C. for 30 seconds, 55° C. for 30 seconds, and 72° C. for 2 minutes was repeated 30 times. After the completion of the reaction, heating was carried out at 72° C. for 10 minutes, and the temperature was reduced to 4° C.

The resulting PCR products were subjected to 1.5% agarose gel electrophoresis, bands were cleaved from the gel, and the cleaved DNAs were purified using the QIAquick Gel Extraction Kit (Qiagen) in accordance with a conventional technique. Purified DNA samples were transformed into E. coli cells (E. coli TOP 10) using the pBAD TOPO TA Expression Kit (Invitrogen). From each of the obtained transformants, a single cell was selectively subjected to streak culture on an LB plate containing 0.5% CM cellulose, and culture was conducted at 37° C. for 18 hours. Thereafter, a 0.2% congo red solution was thinly spread on an agar surface, the resultant was allowed to stand for 15 minutes, the Congo red solution was discarded, a 1 M NaCl solution was spread in the same manner, the resultant was allowed to stand for 20 minutes, and cellulase expression was confirmed based on a clear zone formed in the vicinity of the colony. The E. coli cell transformed with a vector not containing any cellulase gene incorporated therein was subjected to the same procedure, and it was confirmed that a clear zone would not be formed in such cell.

Transformants in which cellulase expression was observed were inoculated into 1 ml of LB medium (containing 100 mg/l ampicillin), pre-shake culture was carried out at 37° C. for 18 hours, 1 ml of the culture solution was added to 100 ml of LB medium (containing 100 mg/l ampicillin), shake culture was conducted until the turbidity (OD 660) reached 0.5, 0.1 ml of 20% L-arabinose solution was added thereto, and culture was conducted again for 4 hours to induce gene expression. After the culture, cells were harvested and washed three times with 0.7% physiological saline. With the use of the Ni-NTA Purification System (Invitrogen), cellulases expressed in accordance with the instructions thereof were purified from the washed cells. Specifically, the washed cells were suspended in 8 ml of Native Binding buffer, 8 mg of chicken egg white lysozyme (Sigma) was added thereto, and the resultant was allowed to stand on ice for 30 minutes. Thereafter, the resultant was ultrasonically treated for 10 seconds, followed by ice-cooling for 10 seconds, and this cell-breaking treatment was repeated 6 times. Thereafter, the resultant was centrifuged at 3,000 G for 15 minutes, and the supernatant was collected. Ni-NTA agarose (1.5 m) was added to the attached column and allowed to spontaneously precipitate for 5 minutes to remove the supernatant. The carrier was washed once with 6 ml of distilled water and twice with 6 ml of the Native Binding buffer. The solution of broken cells (8 ml) was added to the carrier and subjected to slow shaking for 60 minutes, so as to allow target proteins to adsorb onto the carrier. Thereafter, the supernatant was removed via spontaneous precipitation, and the remnant was washed four times with 8 ml of the Native Wash buffer. The Native Elution buffer (8 ml) was then added, 3 ml of the first-eluted portion was collected, and solutions of purified cellulases were obtained.

### Example 2

### Property Analysis of Novel Cellulase

### <Substrate Specificity>

CM cellulose, microcrystalline cellulose (Wako), wheat β-glucan (Sigma), mannan (Sigma), laminarin (Sigma), and phosphoric acid-swollen cellulose were dissolved in 0.1 M phosphate buffer (pH 7.0) to final concentrations of 1% (w/v), respectively, so as to obtain 0.9 ml of a substrate solution. An adequately diluted enzyme solution (0.1 ml) was added thereto, the resultant was subjected to static reaction at 50° C. for 60 minutes (crystalline cellulose was subjected to the reaction with shaking at 50° C. for 18 hours), and enzyme activity was then assayed in the following manner. A DNS (3,5-dinitrosalicylic acid) solution (1 ml) was added to the reaction solution, and the mixture was thermally treated in a boiling water bath for 5 minutes. Thereafter, the resultant was cooled in ice water, 4 ml of deionized water was added, the mixture was agitated, and the absorbance at 535 nm was then assayed using a Hitachi U 1500 spectrophotometer. One enzyme unit was the amount of an enzyme releasing 1 μmol of glucose in 1 minute.

Phosphoric acid-swollen cellulose used was prepared in the following manner. Cellulose powder (100-200 mesh, 5 g, Toyo Roshi Kaisha Ltd.) was suspended in 100 ml of 85% phosphoric acid (Kanto Chemical Co., Inc.), the resultant was allowed to swell at room temperature for 12 hours, and the supernatant was obtained via centrifugation at 10,000×g for 15 minutes. The supernatant was added to 500 ml of distilled water to precipitate non-crystalline cellulose fiber, and the resulting precipitate was collected via centrifugation and then suspended in 500 ml of 0.05% sodium carbonate for neutralization. Thereafter, the precipitate was collected again via centrifugation. The precipitate was suspended in 500 ml of distilled water again and washed (This process was repeated three times), and the resulting precipitate was suspended in 100 ml of 10 mM sodium phosphate (pH 7.0) in the end.

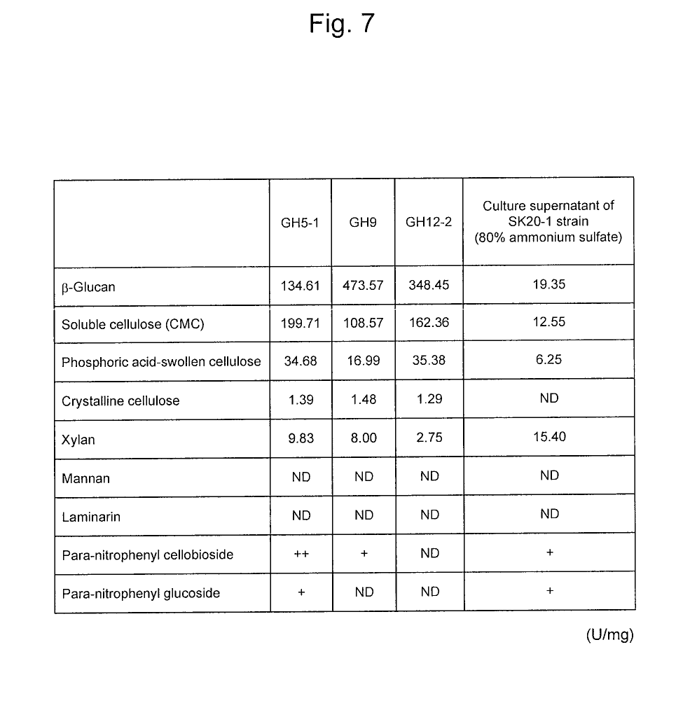

Enzyme activity on various types of substrates is shown in FIG. 7.

All of GH5-1, GH9, and GH12-2 exhibited activity on β-glucan, CM cellulose, microcrystalline cellulose, and xylan. GH5-1 also exhibited activity on para-nitrophenyl cellobioside and para-nitrophenyl glucoside, and GH9 also exhibited activity on para-nitrophenyl cellobioside.

While general bacteria-derived endo-cellulase exhibits substantially no activity on crystalline cellulose and phosphoric acid-swollen cellulose prepared by allowing crystalline cellulose to swell with the aid of acid, all of GH5-1, GH9, and GH12-2 exhibited activity on both substrates (the results of TCL may also be referred to).

In recent years, activity of endo-cellulase derived from Trichoderma viride, which has been generally used at an industrial level, on CM cellulose is known to be about 50 U/mg. Such activity of GH5-1, GH9, and GH12-2 was found to be 2 to 4 times greater than that of such endo-cellulase.

### <Optimal Reaction Temperature>

A substrate solution (1% (w/v) CM cellulose) and 0.1 ml of an adequately-diluted enzyme solution were added to 0.1 M phosphate buffer (pH 7.0), and the reaction was allowed to proceed at 10° C. to 90° C. (the reaction was carried out at temperature intervals of 10° C.). Thereafter, enzyme activity was assayed as described in the “Substrate specificity” section above. The activity exhibiting the maximal value was designated as 100%, and the enzyme activity relative thereto was determined (100% activity: GH5-1: 210 U/mg; GH9: 88 U/mg; GH 12-2: 193 U/mg).

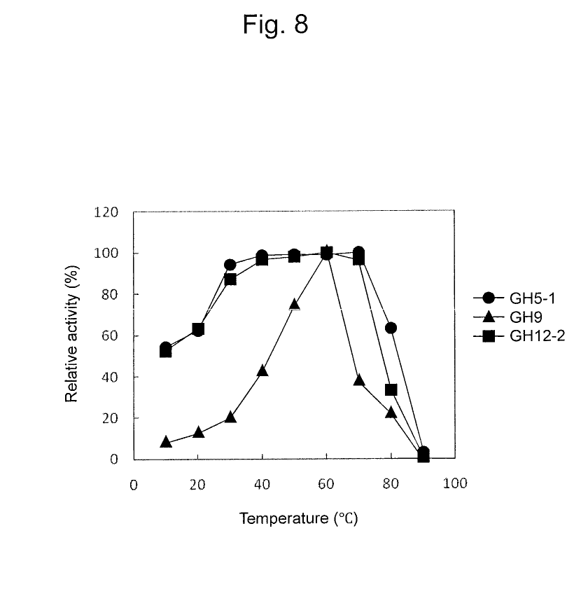

The results are shown in FIG. 8.

GH5-1 exhibited activity of 50% or higher at 10° C. to 80° C., and it was found to be active in a very wide temperature range. As with the case of GH5-1, GH12-2 was found to be active in a very wide temperature range. In contrast, the optimal reaction temperature for GH9 was found to be about 60° C.

<Optimal Reaction pH>

A substrate solution (1% (w/v) CM cellulose) and 0.1 ml of an adequately diluted enzyme solution were added to 0.1 M buffers (i.e., glycine-HCl buffers (pH 2.0, pH 3), citrate-sodium citrate buffers (pH 4, pH 5), phosphate buffers (pH 6.0, pH 7.0), Tris-HCl buffers (pH 8.0, pH 9.0), a glycine-sodium hydroxide buffer (pH 10), and a phosphate-sodium hydroxide buffer (pH 11)), reactions were allowed to proceed at 50° C. for 60 minutes, and enzyme activity was assayed in the manner described in the “Substrate specificity” section above. The activity exhibiting the maximal value was designated as 100%, and the enzyme activity relative thereto was determined (100% activity: GH5-1: 212 U/mg; GH9: 110 U/mg; GH12-2: 177 U/mg).

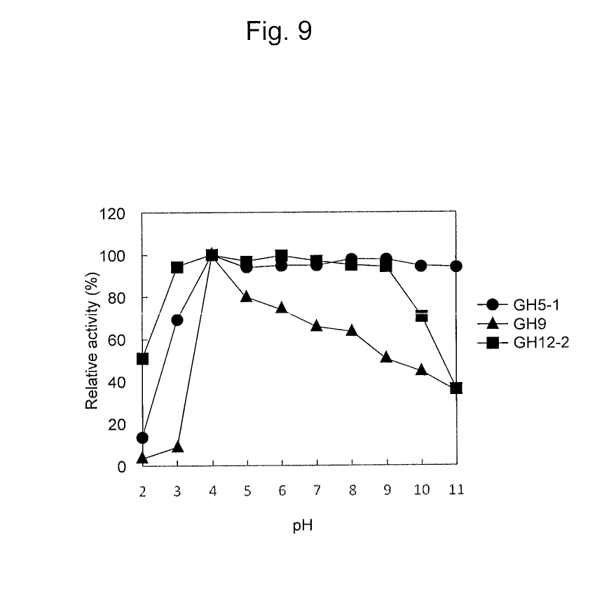

The results are shown in FIG. 9.

GH5-1 and GH12-2 were found to exhibit activity at pH 2 to 11 and to be active in a very extensive pH range. In contrast, GH9 was found to have an optimal pH of 4.

### <Temperature Stability>

Adequately diluted enzyme solutions were thermally treated in 0.1 M phosphate buffer (pH 7.0) at 30° C., 40° C., 50° C., 60° C., 70° C., 80° C., and 90° C. for 10 to 30 minutes, and remaining activity was assayed using 1% (w/v) CM cellulose. Activity assayed without thermal treatment was designated as 100%, and activity remaining relative thereto was determined (100% activity: GH5-1: 252 U/mg; GH9: 103 U/mg; GH12-2: 222 U/mg).

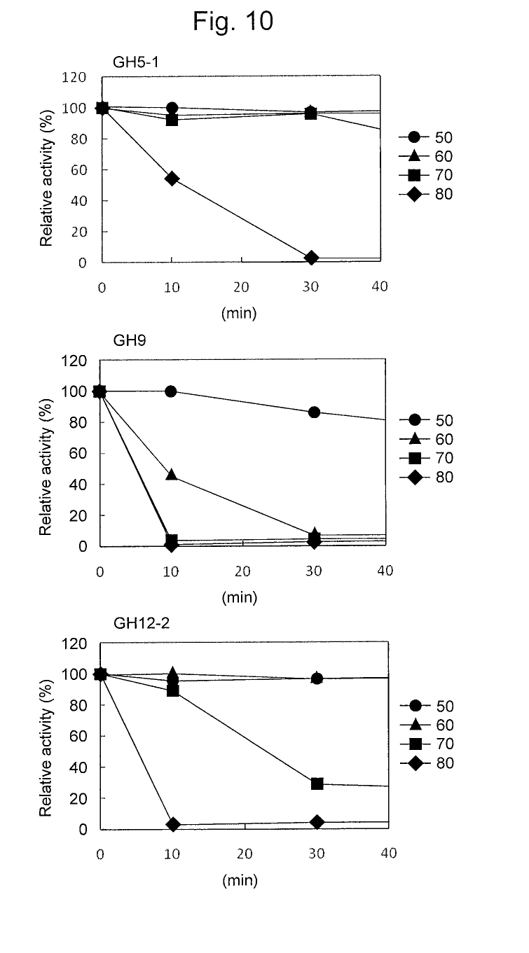

The results are shown in FIG. 10.

GH5-1 was not substantially inactivated even when it was thermally treated at 70° C. for 30 minutes, and it was thus found to be excellent in terms of heat tolerance. In contrast, GH9 was inactivated upon thermal treatment at 70° C. for 10 minutes, and it was thus found to have substantially no heat tolerance. As with the case of GH5-1, GH12-2 was found to be active in a very extensive temperature range. In contrast, GH9 was found to have an optimal reaction temperature of around 60° C.

### <Tolerance to Various Organic Solvents>

A 0.1 M phosphate buffer (pH 7.0) containing 0.1% (w/v) CM cellulose and 25% (v/v) toluene (Kanto Chemical Co., Inc.), acetone (Kanto Chemical Co., Inc.), chloroform (Kanto Chemical Co., Inc.), butanol (Kanto Chemical Co., Inc.), TE saturated phenol (Nippon Gene Co., Ltd.), hexane (Kanto Chemical Co., Inc.), or DMSO (Wako) was used as a substrate solution, the substrate solution was allowed to react with an adequately diluted enzyme solution at 50° C. for 60 minutes, and enzyme activity was assayed in the manner as described in the “Substrate specificity” section above. The activity assayed with the use of an organic-solvent-free substrate solution was designated as 100%, and the activity relative thereto was determined (100% activity: GH5-1: 218 U/mg; GH9: 80 U/mg; GH12-2: 201 U/mg).

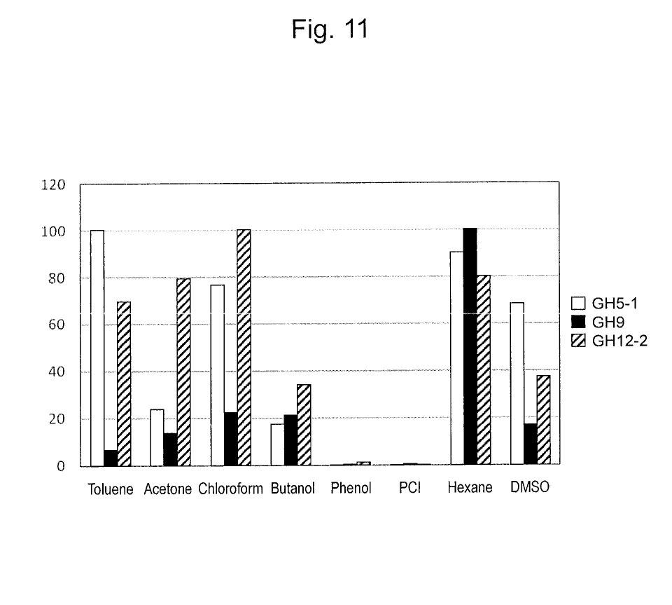

The results are shown in FIG. 11.

GH5-1 and GH12-2 were found to maintain activity in many types of organic solvents. In contrast, GH9 did not exhibit a high level of activity except for in hexane.

### <Ethanol Tolerance>

A 0.1 M phosphate buffer (pH 7.0) containing 0.1% (w/v) CM cellulose and 1%, 3%, 5%, 10%, 20%, 30%, or 50% (v/v) ethanol (Kanto Chemical Co., Inc.) was used as a substrate solution, the substrate solution was allowed to react with an adequately diluted enzyme solution at 50° C. for 60 minutes, and enzyme activity was assayed in the manner as described in the “Substrate specificity” section above. The activity assayed with the use of an ethanol-free substrate solution was designated as 100%, and the activity relative thereto was determined (100% activity: GH5-1: 202 U/mg; GH9: 75 U/mg; GH12-2: 180 U/mg).

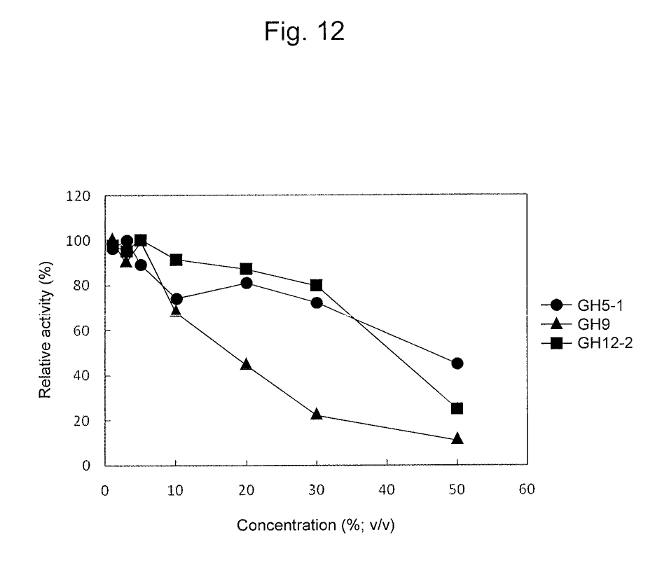

The results are shown in FIG. 12.

GH5-1 and GH12-2 were found to maintain activity in the presence of concentrated ethanol.

### <NaCl Tolerance>

A 0.1 M phosphate buffer (pH 7.0) containing 0.1% (w/v) CM cellulose and 1, 2, 3, 4, or 5 M NaCl was used as a substrate solution, the substrate solution was allowed to react with an adequately diluted enzyme solution at 50° C. for 60 minutes, and enzyme activity was assayed in the manner as described in the “Substrate specificity” section above. The activity assayed with the use of an NaCl-free substrate solution was designated as 100%, and the activity relative thereto was determined (100% activity: GH5-1: 198 U/mg; GH9: 83 U/mg; GH12-2: 180 U/mg).

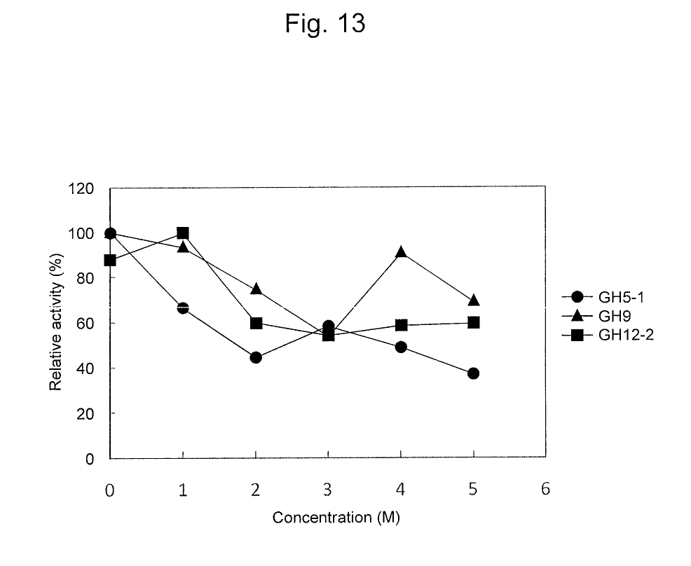

The results are shown in FIG. 13.

GH9 and GH12-2 were found to exhibit relative activity of 50% or more in the presence of 5 M NaCl (about 25% (w/v)) and to be cellulases having very high degrees of salt tolerance.

### <Synergistic Effects of Combination>

Whatman No. 1 filter paper was dissolved in 0.1 M phosphate buffer (pH 7.0) to prepare a substrate solution, and each purified cellulase was adjusted to have a protein concentration of 107 μg/ml. The resulting samples were added to the substrate solution in the following manner. When testing a single type of enzyme, 0.03 ml thereof was added. When testing a mixture of two types of enzymes (i.e., a combination of GH5-1 and GH9, GH5-1 and GH12-2, or GH9 and GH12-2), 0.015 ml each thereof was added (0.03 ml in total). When testing a mixture of three types of enzymes (i.e., a combination of GH5-1, GH9, and GH12—2), 0.01 ml each thereof was added (0.03 ml in total). The reactions were then allowed to proceed at 50° C. for 60 minutes, and enzyme activity was assayed in the manner described in the “Substrate specificity” section above. The theoretical value regarding the enzyme mixture was determined by dividing a sum of measured values of enzymes by the number of enzymes combined. Also, the value indicating the synergistic effects of the enzyme mixture was determined by dividing the measured value of the enzyme mixture by the theoretical value.

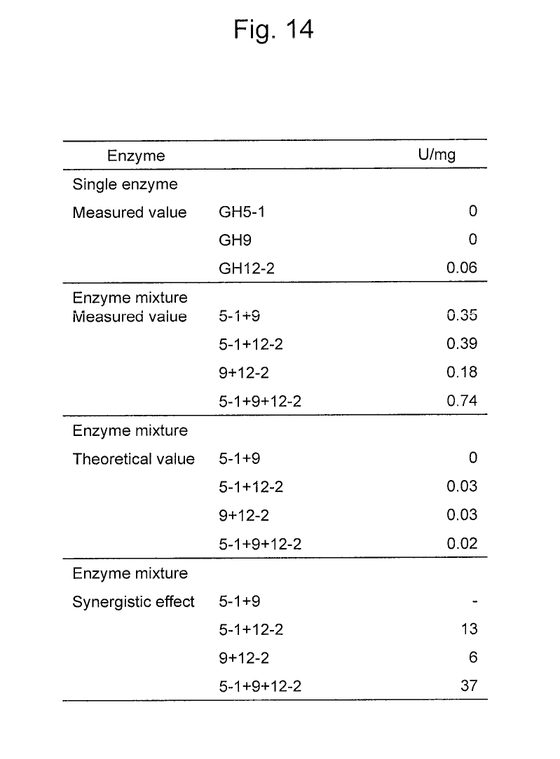

The results are shown in FIG. 14.

Enzyme activity of GH5-1, GH9, and GH12-2 was found to improve in a synergistic manner with the use of such enzymes in combinations of two or more.

### <Degradation Property Analysis Via TLC>

CM cellulose, microcrystalline cellulose (Wako), filter paper (Whatman No. 1), wheat β-glucan, phosphoric acid-swollen cellulose, cellobiose (Yaizu Suisankagaku Industry Co., Ltd.), cellotriose (Yaizu Suisankagaku Industry Co., Ltd.), cellotetraose (Yaizu Suisankagaku Industry Co., Ltd.), and cellopentaose (Yaizu Suisankagaku Industry Co., Ltd.) were each dissolved in 0.1 M phosphate buffer (pH 7.0) to a final concentration of 1% (w/v), so as to obtain 0.9 ml of a substrate solution. An adequately diluted enzyme solution (0.1 ml) was added thereto, the resultant was subjected to static reaction at 50° C. for 60 minutes (crystalline cellulose and filter paper were subjected to the reaction with shaking at 50° C. for 18 hours), 20 μl of the supernatant was spotted on a thin-layer plate (TLC silica gel 60, Merck), and the resultant was soaked in a developing solution (chloroform:acetic acid:water=6:7:1) to analyze the degradation product.

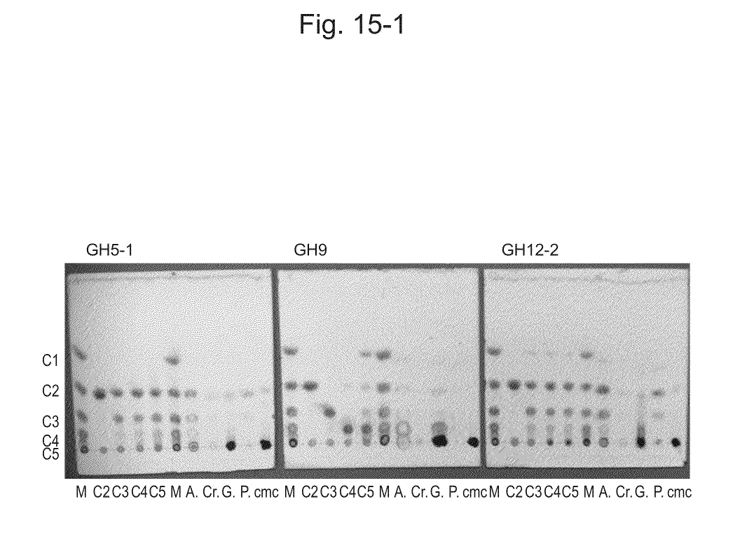

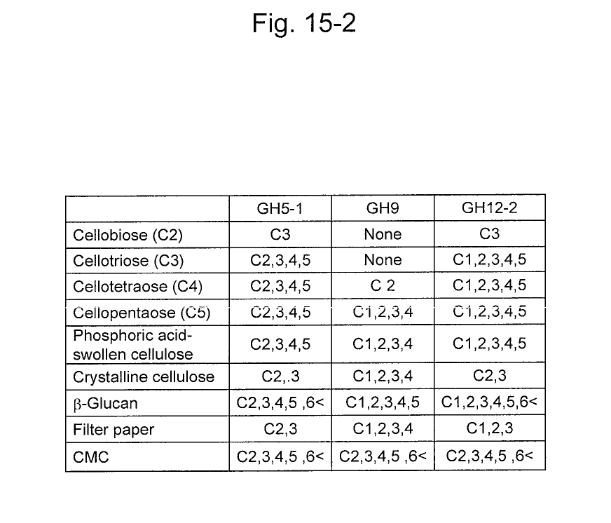

The results are shown in FIGS. 15-1 and 15-2.

Cellotriose (C3) was the minimal degradation unit of both GH5-1 and GH12-2. In degradation products of crystalline cellulose, acid-swollen cellulose, filter paper, and glucan, cellobiose was mainly detected. Glucose was not detected only in GH5-1.

Meanwhile, cellotetraose was the minimal degradation unit of GH9. Cellotetraose was mainly detected in degradation products of acid-swollen cellulose, filter paper, and glucan.

Both GH5-1 and GH12-2 exhibited transgrlycolation activity. When GH5-1 and GH12-2 were allowed to react with a trisaccharide or tetrasaccharide, they were capable of producing oligosaccharides with chains as long as or longer than such saccharides because of the transgrlycolation activity thereof.

### INDUSTRIAL APPLICABILITY

The present invention can provide a novel cellulase derived from the Thermosporothrix hazakensis SK20-1^{T} strain (JCM 16142T=ATCC BAA-1881T). Such cellulase has distinctive features, such as tolerance to various organic solvents, ethanol, and NaCl, and it is expected to make a contribution in industrial fields such as the fine chemical industry (e.g., synthesis of sucrose fatty acid ester), saccharification and alcohol fermentation of biomass materials, and the like.

All publications, patents, and patent applications cited herein are incorporated herein by reference in their entirety.

1. A cellulase derived from Thermosporothrix hazakensis having enzyme activity on at least β-glucan, soluble cellulose, crystalline cellulose, phosphoric acid-swollen cellulose, and xylan.

2. The cellulase according to claim 1, wherein the Thermosporothrix hazakensis is the Thermosporothrix hazakensis SK20-1^{T} strain (JCM 16142T=ATCC BAA-1881T).

3. The cellulase according to claim 1 or 2, which retains enzyme activity at a temperature of at least 10° C. to 80° C.

4. The cellulase according to claim 1 or 2, which retains enzyme activity at a pH of at least 2 to 11.

5. The cellulase according to claim 1 or 2, which retains enzyme activity in the presence of an organic solvent at 0% to 25% (v/v) or higher concentration.

6. The cellulase according to claim 5, wherein the organic solvent is selected from the group consisting of toluene, acetone, chloroform, butanol, hexane, and DMSO.

7. The cellulase according to claim 1 or 2, which retains enzyme activity in the presence of ethanol at 0% to 50% (v/v) or higher concentration.

8. The cellulase according to claim 1 or 2, which retains enzyme activity in the presence of salt at 0% to 25% (v/v) or higher concentration.

9. The cellulase according to claim 1 or 2, which comprises one or more hydrolases selected from the group consisting of hydrolases comprising polypeptides represented by the amino acid sequences below:
(I) a polypeptide comprising the amino acid sequence as shown in SEQ ID NO: 1, a polypeptide comprising an amino acid sequence having deletion, substitution, insertion, or addition of one or several amino acids in the amino acid sequence as shown in SEQ ID NO: 1 and having cellulase activity, or a polypeptide comprising an amino acid sequence having at least 90% identity with the amino acid sequence as shown in SEQ ID NO: 1 and having cellulase activity;
(II) a polypeptide comprising the amino acid sequence as shown in SEQ ID NO: 2, a polypeptide comprising an amino acid sequence having deletion, substitution, insertion, or addition of one or several amino acids in the amino acid sequence as shown in SEQ ID NO: 2 and having cellulase activity, or a polypeptide comprising an amino acid sequence having at least 90% identity with the amino acid sequence as shown in SEQ ID NO: 2 and having cellulase activity;
(III) a polypeptide comprising the amino acid sequence as shown in SEQ ID NO: 3, a polypeptide comprising an amino acid sequence having deletion, substitution, insertion, or addition of one or several amino acids in the amino acid sequence as shown in SEQ ID NO: 3 and having cellulase activity, or a polypeptide comprising an amino acid sequence having at least 90% identity with the amino acid sequence as shown in SEQ ID NO: 3 and having cellulase activity;
(IV) a polypeptide comprising the amino acid sequence as shown in SEQ ID NO: 4, a polypeptide comprising an amino acid sequence having deletion, substitution, insertion, or addition of one or several amino acids in the amino acid sequence as shown in SEQ ID NO: 4 and having cellulase activity, or a polypeptide comprising an amino acid sequence having at least 90% identity with the amino acid sequence as shown in SEQ ID NO: 4 and having cellulase activity;
(V) a polypeptide comprising the amino acid sequence as shown in SEQ ID NO: 5, a polypeptide comprising an amino acid sequence having deletion, substitution, insertion, or addition of one or several amino acids in the amino acid sequence as shown in SEQ ID NO: 5 and having cellulase activity, or a polypeptide comprising an amino acid sequence having at least 90% identity with the amino acid sequence as shown in SEQ ID NO: 5 and having cellulase activity; and
(VI) a polypeptide comprising the amino acid sequence as shown in SEQ ID NO: 6, a polypeptide comprising an amino acid sequence having deletion, substitution, insertion, or addition of one or several amino acids in the amino acid sequence as shown in SEQ ID NO: 6 and having cellulase activity, or a polypeptide comprising an amino acid sequence having at least 90% identity with the amino acid sequence as shown in SEQ ID NO: 6 and having cellulase activity.

10. A polynucleotide encoding the cellulase according to any one of claims 1 to 2.

11. An expression vector comprising the polynucleotide according to claim 10.

12. A transformant obtained with the use of the expression vector according to claim 11.

13. A culture product obtained by culturing the transformant according to claim 11.

14. A detergent composition comprising the cellulase according to any one of claims 1 to 2.

15. A method for saccharification of a carbohydrate-containing raw material comprising treating a carbohydrate-containing raw material with a cellulase derived from Thermosporothrix hazakensis having enzyme activity on at least β-glucan, soluble cellulose, crystalline cellulose, phosphoric acid-swollen cellulose, and xylan, a polynucleotide encoding the cellulose, an expression vector comprising the polynucleotide, a transformant obtained with the use of the expression vector or a culture product obtained by culturing the transformant.

16. A method for producing a food or feed product comprising treating a carbohydrate-containing raw material with a cellulase derived from Thermosporothrix hazakensis having enzyme activity on at least β-glucan, soluble cellulose, crystalline cellulose, phosphoric acid-swollen cellulose, and xylan, a polynucleotide encoding the cellulose, an expression vector comprising the polynucleotide, a transformant obtained with the use of the expression vector or a culture product obtained by culturing the transformant.

17. A method for producing ethanol comprising:
(i) treating a carbohydrate-containing raw material with a cellulase derived from Thermosporothrix hazakensis having enzyme activity on at least β-glucan, soluble cellulose, crystalline cellulose, phosphoric acid-swollen cellulose, and xylan, a polynucleotide encoding the cellulose, an expression vector comprising the polynucleotide, a transformant obtained with the use of the expression vector or a culture product obtained by culturing the transformant; and
(ii) subjecting the product obtained in step (i) to fermentation.

18. A detergent composition comprising the cellulase according to the culture product according to claim 13.

19. The method for saccharification of claim 15 wherein the Thermosporothrix hazakensis is the Thermosporothrix hazakensis SK20-1T strain (JCM 16142T=ATCC BAA-1881T).

20. The method for producing a food or feed product of claim 16, wherein the Thermosporothrix hazakensis is the Thermosporothrix hazakensis SK20-1T strain (JCM 16142T=ATCC BAA-1881T).

21. A method for producing ethanol of claim 17, wherein the Thermosporothrix hazakensis is the Thermosporothrix hazakensis SK20-1T strain (JCM 16142T=ATCC BAA-1881T).
